(19) United States
(12) Patent Application Publication
(10) Pub. No.: US 2025/0382483 A1
(43) Pub. Date: Dec. 18, 2025

(54) ADDITIVE MANUFACTURING OF DENTAL PROSTHESES

(71) Applicant: Stratasys Ltd., Rehovot (IL)

(72) Inventors: Yaniv HIRSCHSOHN, Rehovot (IL); Inna VINTS, Rehovot (IL); Dani PERI, Rehovot (IL); Elena SHPAYZER, Gedera (IL); Lior ZONDER, Tel-Aviv (IL)

(73) Assignee: Stratasys Ltd., Rehovot (IL)

(21) Appl. No.: 19/303,401

(22) Filed: Aug. 19, 2025

Related U.S. Application Data
(63) Continuation of application No. 18/755,764, filed on Jun. 27, 2024.
Application No. 18/755,764 is a continuation of application No. PCT/IL2022/051414, filed on Dec. 29, 2022.
(60) Provisional application No. 63/295,639, filed on Dec. 31, 2021.

(51) Int. Cl. C09D 11/322; A61C 13/00; B29C 64/112; B29K 33/00; B29K 105/00; B29K 105/16; B29K 507/00; B29L 31/00; B33Y 10/00; B33Y 70/00; B33Y 80/00; C09D 11/037; C09D 11/107

(52) U.S. Cl. CPC C09D 11/322; A61C 13/0019; B33Y 70/00; B33Y 80/00; C09D 11/037; C09D 11/107; B29C 64/112; B29K 2033/00; B29K 2039/00; B29K 2105/0032; B29K 2105/16; B29K 2507/00; B29L 2031/7536; B33Y 10/00

(57) ABSTRACT

Modeling material formulations usable in additive manufacturing of a denture structure, and additive manufacturing methods employing same are provided. Kits comprising the modeling material formulation, optionally in combination with a support material formulation, are also provided.

### RELATED APPLICATIONS

This application is a Continuation of U.S. patent application Ser. No. 18/755,764, filed on Jun. 27, 2025, which is a Continuation of PCT Patent Application No. PCT/IL2022/051414 having International Filing Date of Dec. 29, 2022, which claims the benefit of priority under 35 USC § 119 (e) of U.S. Provisional Patent Application No. 63/295,639 filed on Dec. 31, 2021. The contents of the above applications are all incorporated by reference as if fully set forth herein in their entirety.

### FIELD AND BACKGROUND OF THE INVENTION

The present invention, in some embodiments thereof, relates to additive manufacturing and, more particularly, but not exclusively, to curable formulations which are usable in additive manufacturing of dental prostheses, including denture teeth, denture base and monolithic denture structures.

Additive manufacturing (AM) is a technology enabling fabrication of arbitrarily shaped structures directly from computer data via additive formation steps. The basic operation of any AM system consists of slicing a three-dimensional computer model into thin cross sections, translating the result into two-dimensional position data and feeding the data to control equipment which fabricates a three-dimensional structure in a layer-wise manner.

Additive manufacturing entails many different approaches to the method of fabrication, including three-dimensional (3D) printing such as 3D inkjet printing, electron beam melting, stereolithography, selective laser sintering, laminated object manufacturing, fused deposition modeling and others.

Some 3D printing processes, for example, 3D inkjet printing, are being performed by a layer by bayer inkjet deposition of building materials. Thus, a building material is dispensed from a dispensing head having a set of nozzles to deposit layers on a supporting structure. Depending on the building material, the layers may then be cured or solidified. Curing may be by exposure to a suitable condition, and optionally by using a suitable device.

The building material includes an uncured model material (also referred to as “uncured modeling material” or “modeling material formulation”), which is selectively dispensed to produce the desired object, and may also include an uncured support material (also referred to as “uncured supporting material” or “support material formulation”) which provides temporary support to specific regions of the object during building and assures adequate vertical placement of subsequent object layers. The supporting structure is configured to be removed after the object is completed.

In some known inkjet printing systems, the uncured model material is a photopolymerizable or photocurable material that is cured, hardened or solidified upon exposure to ultraviolet (UV) light after it is jetted. The uncured model material may be a photopolymerizable material formulation that has a composition which, after curing, gives a solid material with mechanical properties that permit the building and handling of the three-dimensional object being built. The modeling material formulation typically include a reactive (curable) component and a photo-initiator. The photo-initiator may enable at least partial solidification (hardening) of the uncured support material by curing with the same UV light applied to form the model material. The solidified material may be rigid, or may have elastic properties.

The support material is formulated to permit fast and easy cleaning of the object from its support. The support material may be a polymer, which is water-soluble and/or capable of swelling and/or breaking down upon exposure to a liquid solution, e.g. water, alkaline or acidic water solution. The support material formulation may also include a reactive (curable) component and a photo-initiator.

In order to be compatible with most of the commercially-available print heads utilized in a 3D inkjet printing system, the uncured building materials should feature the following characteristics: a relatively low viscosity (e.g., Brookfield Viscosity of up to 50 centipoises or cps, or up to 35 cps, preferably from 8 to 25 cps) at the working (e.g., jetting) temperature; Surface tension of from about 25 to about 55 Dyne/cm, preferably from about 25 to about 40 Dyne/cm; and a Newtonian liquid behavior and high reactivity to a selected curing condition, to enable fast solidification of the jetted layer upon exposure to a curing condition, of no more than 1 minute, preferably no more than 20 seconds.

The hardened modeling material which forms the final object typically exhibits a heat deflection temperature (HDT) which is higher than room temperature, in order to assure its usability. Desirably, the hardened modeling material exhibits an HDT of at least 35° C. For an object to be stable at variable conditions, a higher HDT is known to be desirable. In most cases, it is also desirable that the object exhibits relatively high Izod Notched impact, e.g., higher than 50 or higher than 60 J/m.

Various three-dimensional printing techniques exist and are disclosed in, e.g., U.S. Pat. Nos. 6,259,962, 6,569,373, 6,658,314, 6,850,334, 6,863,859, 7,183,335, 7,209,797, 7,225,045, 7,300,619, 7,500,846, 7,991,498 and 9,031,680 and U.S. Published application No. 20160339643, all by the same Assignee, and being hereby incorporated by reference in their entirety.

Several additive manufacturing processes, including three-dimensional inkjet printing, allow additive formation of objects using more than one modeling material, also referred to as “multi-material” AM processes. For example, U.S. Patent Application having Publication No. 2010/0191360, of the present Assignee, discloses a system which comprises a solid freeform fabrication apparatus having a plurality of print heads, a building material supply apparatus configured to supply a plurality of building materials to the fabrication apparatus, and a control unit configured for controlling the fabrication and supply apparatus. The system has several operation modes. In one mode, all print heads operate during a single building scan cycle of the fabrication apparatus. In another mode, one or more of the print heads is not operative during a single building scan cycle or part thereof.

In a 3D inkjet printing process such as Polyjet™ (Stratasys® Ltd., Israel), the building material is selectively jetted from one or more inkjet print heads and/or nozzles and deposited onto a fabrication tray in consecutive layers according to a pre-determined configuration as defined by a software file.

The Polyjet™ technology allows control over the position and composition of each voxel (volume pixel), which affords enormous design versatility and digital programming of multi-material structures. Other advantages of the Polyjet™ technology is the very high printing resolution, up to 14 μm layer height, and the ability to print multiple materials simultaneously, in a single object. This multi-material 3D printing process often serves for fabrication of complex parts and structures that are comprised of elements having different stiffness, performance, color or transparency. New range of materials, programmed at the voxel level, can be created by the PolyJet™ printing process, using only few starting materials.

International Patent Application Publication No. WO 2013/128452, by the present Assignee, discloses a multi-material approach which involves separate jetting of two components of a cationic polymerizable system and/or a radical polymerizable system, which intermix on the printing tray, leading to a polymerization reaction similar to pre-mixing of the two components before jetting, while preventing their early polymerization on the inkjet head nozzle plate.

Current PolyJet™ technology offers the capability to use a range of curable (e.g., polymerizable) materials that provide polymeric materials featuring a variety of properties, ranging, for example, from stiff and hard materials (e.g., curable formulations marketed as the Vero™ Family materials) to soft and flexible materials (e.g., curable formulations marketed as the Tango™ and Agilus™ families), and including also objects made using Digital ABS, which contain a multi-material made of two starting materials (e.g., RGD515™ & RGD535/531™), and simulate properties of engineering plastic. Most of the currently practiced PolyJet™ materials are curable materials which harden or solidify upon exposure to radiation, mostly UV radiation and/or heat, with the most practiced materials being acrylic-based materials.

Some photocurable (photopolymerizable) modeling material formulations known as usable in 3D inkjet printing are designed so as to provide, when hardened, a transparent material.

The use of light emitting diodes (LED) as a source for electromagnetic irradiation has recently become more and more common and desirable in many fields, including additive manufacturing processes such as those that utilize UV-curable materials. Most of the commercially available UV LED light sources emit UVA radiation, at the higher wavelengths of 365/395/405 nm. The use of such light sources poses severe limitations since photoinitiators that absorb shorter wavelength, such as, for example, those of the alpha-hydroxy ketone family that absorb at 250-300 nm, cannot be efficiently used. These photoinitiators are typically used for surface curing and the absence thereof adversely affect the process quality.

Current solutions to the limitations posed by the use of UV LED as an irradiation source include the use of hydrogen donors that promote surface curing, such as tertiary amines, thiols and polyethylene glycol-containing materials. However, the use of these materials, while facilitating AM that use UV LED, is accompanied by several drawbacks. For example, tertiary amines impart an increased yellow hue to the cured material; thiols are typically reactive towards UV-curable materials that are commonly used in AM, such as acrylic materials, and thus limit the shelf-lives of formulations containing same; and polyethylene glycol materials are amphiphilic materials that act also as plasticizers or elastomers and hence reduce mechanical stability and increase water absorption of the obtained object.

During the last decade, efforts have been made to use additive manufacturing such as 3D inkjet printing and digital light processing (DLP) in the denture field.

For example, U.S. Pat. No. 7,476,347 and U.S. Patent Application Publication No. 2011/0049738 disclose a process for making dentures having integral teeth and a denture base by inkjet three-dimensional printing. The methodologies taught in these patents employ wax-like polymerizable materials, which are needed to be custom-synthesized, incurring additional time and costs. These materials require the use of more than 70% filler material, and feature slow reaction rate and high viscosity.

U.S. patent application No. 20190175455 describes a photocurable composition for manufacturing a dental prosthesis by stercolithography, including: a photopolymerization initiator; and a (meth)acrylic monomer component including an acrylic monomer (X) having no aromatic rings and having a ring structure other than an aromatic ring and two or more acryloyloxy groups in one molecule and having an Mw of from 200 to 800, and at least one of a (meth)acrylic monomer (A) having one or more ether bonds and two (meth)acryloyloxy groups in one molecule and having a defined Mw, a (meth)acrylic monomer (B) having a ring structure other than an aromatic ring and one (meth)acryloyloxy group in one molecule and having a defined Mw, a (meth)acrylic monomer (C) having a hydrocarbon skeleton and two (meth)acryloyloxy groups in one molecule and having a defined Mw, and a (meth)acrylic monomer (D) having one or more aromatic rings and one (meth)acryloyloxy group in one molecule and having a Mw.

U.S. Patent Application Publication No. 20180049954 teaches photo-curable compositions for artificial teeth and denture base which are usable in 3D inkjet printing or DLP type AM. The compositions include photo-curable organic compounds, surface modified nano-sized inorganic filler, photo-initiator, colorant, and stabilizer. The compositions provide a distinctive denture base and a set of artificial teeth which can thereafter be bonded to one another.

Additional background art includes Chung et al., Materials (Basel). 2018 October; 11(10): 1798; and U.S. Pat. Nos. 9,227,365; 6,242,149; U.S. Patent Application having Publication No. 2010/0140850; WO 2009/013751; WO 2016/063282; WO 2016/125170; WO 2017/134672; WO 2017/134673; WO 2017/134674; WO 2017/134676; WO 2017/068590; WO 2017/187434; WO 2018/055521; WO 2018/055522; and WO 2020/065654; all by the present assignee.

### SUMMARY OF THE INVENTION

According to an aspect of some embodiments of the present invention there is provided a modeling material formulation usable in additive manufacturing of a denture structure, the modeling material formulation comprising:
a multi-functional (e.g., di-functional) aliphatic urethane (meth)acrylate featuring, when hardened, Tg higher than 100° C. (Component A);
a multi-functional (e.g., di-functional) non-aromatic (meth)acrylate featuring, when hardened, Tg higher than 100° C. (Component B);
a filler in a form of micron-sized particles (Component C);
a multi-functional (e.g., di-functional) ethoxylated aromatic (meth)acrylate featuring less than 10 ethoxylated groups and/or featuring, when hardened, Tg that ranges from 50 to 150° C. (Component D);
a mono-functional (meth)acrylate (Component E);
a multi-functional (e.g., tri-functional) cyclic (meth)acrylate (Component F); and
a multi-functional (e.g., di-functional) aliphatic urethane (meth)acrylate featuring, when hardened, Tg lower than 100° C. (Component G),
wherein:
an amount of the filler is no more than 20, or no more than 15, % by weight of the total weight of the formulation; and
an amount of the Component D is no more than 20, or no more than 15, % by weight of the total weight of the formulation.

According to some of any of the embodiments described herein, an amount of the Component A ranges from 15 to 25, % by weight of the total weight of the formulation.

According to some of any of the embodiments described herein, an amount of the component B is no more than 20, or no more than 15, % by weight of the total weight of the formulation.

According to some of any of the embodiments described herein, a total amount of the Component B and the Component C ranges from about 15 to about 25, % by weight of the total weight of the formulation.

According to some of any of the embodiments described herein, the Component B comprises a multi-functional (e.g., di-functional) alicyclic (meth)acrylate.

According to some of any of the embodiments described herein, the filler particles comprise silica particles.

According to some of any of the embodiments described herein, the filler particles have a plurality of curable groups attached thereto.

According to some of any of the embodiments described herein, the Component D is a multi-functional (e.g., di-functional) ethoxylated aromatic (meth)acrylate featuring less than 5 ethoxylated groups.

According to some of any of the embodiments described herein, the Component D is a multi-functional (e.g., di-functional) ethoxylated aromatic (meth)acrylate featuring less than 5 ethoxylated groups and featuring, when hardened, Tg that ranges from 50 to 150° C.

According to some of any of the embodiments described herein, the mono-functional (meth)acrylate (Component E) comprises a mono-functional acrylate and a mono-functional methacrylate.

According to some of any of the embodiments described herein, a weight ratio of the mono-functional acrylate and the mono-functional methacrylate ranges from 2:1 to 1:2.

According to some of any of the embodiments described herein, a concentration of each of the of the mono-functional acrylate and the mono-functional methacrylate independently ranges from 10 to 20, or from 15 to 20, % by weight of the total weight of the formulation.

According to some of any of the embodiments described herein, a total amount of the at least one mono-functional (meth)acrylate ranges from 30 to 40% by weight of the total weight of the formulation.

According to some of any of the embodiments described herein, the Component F is a multi-functional (e.g., tri-functional) isocyanurate (meth)acrylate.

According to some of any of the embodiments described herein, the Component F features, when hardened, Tg higher than 150, or higher than 180, or higher than 200,° C.

According to some of any of the embodiments described herein, a concentration or an amount of the Component F ranges from 5 to 10, % by weight of the total weight of the formulation.

According to some of any of the embodiments described herein, the Component G is a multi-functional (e.g., di-functional) aliphatic urethane (meth)acrylate, having an average MW of at least 1,000 grams/mol.

According to some of any of the embodiments described herein, a concentration or an amount of the Component G ranges from 5 to 10, % by weight of the total weight of the formulation.

According to some of any of the embodiments described herein, the formulation comprises: the Component A in an amount that ranges from 15 to 25, % by weight of the total weight of the formulation;
the Component B in an amount of no more than 20, or no more than 15, % by weight of the total weight of the formulation;
the Component E is an amount of from 30 to 40% by weight of the total weight of the formulation;
the Component F in an amount of from 5 to 10, % by weight of the total weight of the formulation; and
the Component G in an amount of from 5 to 10, % by weight of the total weight of the formulation.

According to some of any of the embodiments described herein, the Component A is a di-functional aliphatic urethane methacrylate featuring, when hardened, Tg higher than 100° C.

According to some of any of the embodiments described herein, the Component B is a di-functional alicyclic acrylate featuring, when hardened, Tg higher than 100° C.

According to some of any of the embodiments described herein, the Component C comprises micron-sized silica particles having curable groups attached thereto.

According to some of any of the embodiments described herein, the Component D is a di-functional ethoxylated aromatic methacrylate featuring less than 5 ethoxylated groups and, when hardened, Tg that ranges from 50 to 150° C. (Component D).

According to some of any of the embodiments described herein, the Component E comprises a mono-functional acrylate and a mono-functional methacrylate, each independently in an amount of from 10 to 20, or from 15 to 20, % by weight, of the total weight of the formulation.

According to some of any of the embodiments described herein, the Component F is a tri-functional isocyanurate triacrylate.

According to some of any of the embodiments described herein, the Component G is a di-functional aliphatic urethane dimethacrylate featuring, when hardened, Tg lower than 100° C. and an average MW of at least 1,000 grams/mol.

According to some of any of the embodiments described herein, the formulation further comprises a dispersant (Component H).

According to some of any of the embodiments described herein, the dispersant has curable groups.

According to some of any of the embodiments described herein, the dispersant is a multi-functional (e.g., di-functional) aliphatic silicon (meth)acrylate.

According to some of any of the embodiments described herein, the dispersant has an average MW of at least 1,000 grams/mol.

According to some of any of the embodiments described herein, an amount of the dispersant ranges from 0.1 to 0.5, % by weight, of the total weight of the formulation.

According to some of any of the embodiments described herein, the formulation further comprises an inhibitor (Component I).

According to some of any of the embodiments described herein, the formulation further comprises a photoinitiator (Component J).

According to some of any of the embodiments described herein, an amount of the photoinitiator ranges from 1 to 5, % by weight of the total weight of the formulation.

According to some of any of the embodiments described herein, the formulation further comprises a coloring agent (Component P).

According to some of any of the embodiments described herein, the coloring agent comprises a pigment.

According to some of any of the embodiments described herein, the coloring agent comprises a mixture of a pigment and at least one (meth)acrylic material.

According to some of any of the embodiments described herein, the pigment comprises nano-sized particles.

According to some of any of the embodiments described herein, the coloring agent further comprises a pigment dispersant (Component Dp).

According to an aspect of some embodiments of the present invention there is provided a support material formulation usable in additive manufacturing of a denture structure, preferably in combination with the modeling material formulation of any one of claims 1 to 39, the support material formulation comprising:
a non-curable water-soluble or water-miscible polymeric material, in an amount of from about 40 to about 60% by weight of the total weight of the formulation;
a hydrophilic mono-functional (meth)acrylate, in an amount of from 15 to 25% by weight of the total weight of the formulation;
a hydrophilic mono-functional (meth)acrylamide in an amount of from 10 to 20% by weight of the total weight of the formulation; and
a multi-functional non-aromatic (e.g., aliphatic or alicyclic) (meth)acrylate in an amount of from 1 to 5% by weight of the total weight of the formulation.

According to some of any of the embodiments described herein, the support formulation further comprises a photoinitiator, in an amount of from 0.1 to 1% by weight of the total weight of the formulation.

According to an aspect of some embodiments of the present invention there is provided a kit comprising at least one modeling material formulation as described in any of the respective embodiments and any combination thereof, and the support material formulation as described in any of the respective embodiments and any combination thereof, each formulation being packaged individually within the kit.

According to an aspect of some embodiments of the present invention there is provided a kit comprising at least two modeling material formulations as described in any of the respective embodiments and any combination thereof, each individually packaged within the kit, wherein the at least two formulations differ from one another by the presence and/or type of a coloring agent.

According to an aspect of some embodiments of the present invention there is provided a method of additive manufacturing a three-dimensional denture object, the method comprising dispensing a plurality of layers in a configured pattern correspond to the shape the denture object, thereby forming the object, wherein the formation of each of at least a few of the layers comprises dispensing at least one formulation, and exposing the dispensed formulation to a curing condition to thereby form a cured modeling material, wherein the at least one formulation is the modeling material formulation as described in any of the respective embodiments and any combination thereof.

According to some of any of the embodiments described herein, the dispensing is of at least two modeling material formulations, each independently being a formulation as described in any of the respective embodiments and any combination thereof.

According to some of any of the embodiments described herein, the dispensing is further of a support material formulation.

According to some of any of the embodiments described herein, the support material formulation is as described herein in any of the respective embodiments and any combination thereof.

According to some of any of the embodiments described herein, the denture structure is selected from denture base, an artificial tooth, artificial teeth and a monolithic structure of a denture base and artificial teeth.

According to some of any of the embodiments described herein, the denture structure is a monolithic structure of a denture base and artificial teeth.

According to an aspect of some embodiments of the present invention there is provided a denture structure obtained by the AM method as described in any of the respective embodiments and any combination thereof.

According to some of any of the embodiments described herein, the denture structure is a monolithic structure of a denture base and artificial teeth.

According to some of any of the embodiments described herein, the denture structure features mechanical and physical properties in accordance with the requirements of ISO 20795-1 and ISO 10477 and biocompatibility properties in accordance with the requirements of ISO 10993-1.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fec.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D are schematic illustrations of an additive manufacturing system according to some embodiments of the invention;

FIGS. 2A-2C are schematic illustrations of printing heads according to some embodiments of the present invention;

FIGS. 3A and 3B are schematic illustrations demonstrating coordinate transformations according to some embodiments of the present invention;

FIG. 4 is a photograph showing exemplary objects prepared according to some embodiments of the present invention, and particularly a monolithic denture structure as described herein.

FIG. 5 presents comparative plots showing a change in the mechanical properties of an object made of an exemplary modeling material formulation IX as described herein, measured in accordance with ISO 20795-1.

FIGS. 6A-B is a bar graph (FIG. 6B) showing dimensional changes over time, measured in accordance with ISO 20795-1, of hardened model materials taken from selected regions as shown in FIG. 6A, of an internal portion of a monolithic denture as presented in FIG. 4.

FIGS. 7A-B is a bar graph (FIG. 7B) showing dimensional changes over time, measured in accordance with ISO 20795-1, of hardened model materials taken from selected regions as shown in FIG. 7A, of an external portion of a monolithic denture as presented in FIG. 4.

### DESCRIPTION OF SPECIFIC EMBODIMENTS OF THE INVENTION

The present invention, in some embodiments thereof, relates to additive manufacturing and, more particularly, but not exclusively, to curable formulations which are usable in additive manufacturing of dental prostheses, including denture teeth, denture base and monolithic denture structures.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Herein throughout, the term “object” describes a final product of the additive manufacturing. This term refers to the product obtained by a method as described herein, after removal of the support material, if such has been used as part of the uncured building material, and/or after post treatment (e.g., photobleaching such as described herein).

The term “object” as used herein throughout refers to a whole object or a part thereof.

Herein throughout, the phrase “cured modeling material” which is also referred to herein as “hardened” or “solidified” modeling material describes the part of the building material that forms the object, as defined herein, upon exposing the dispensed building material to a curing condition (and optionally post-treatment), and, optionally, if a support material has been dispensed, upon removal of the cured support material, as described herein. The hardened modeling material can be a single hardened material or a mixture of two or more hardened materials, depending on the modeling material formulations used in the method, as described herein.

The phrases “cured modeling material”, “hardened modeling material”, “solidified modeling material” or “cured/hardened/solidified modeling material formulation” can be regarded as a cured building material wherein the building material consists only of a modeling material formulation (and not of a support material formulation). That is, this phrase refers to the portion of the building material, which is used to provide the final object.

Herein throughout, the phrase “modeling material formulation”, which is also referred to herein interchangeably as “modeling formulation”, “modeling material” “model material” or simply as “formulation”, describes a part or all of the uncured building material which is dispensed so as to form the object, as described herein. The modeling material formulation is an uncured modeling formulation (unless specifically indicated otherwise), which, upon exposure to a condition that effects curing, may form the object or a part thereof.

In some embodiments of the present invention, a modeling material formulation is formulated for use in three-dimensional inkjet printing and is able to form a three-dimensional object on its own, i.e., without having to be mixed or combined with any other substance.

An uncured building material can comprise one or more modeling material formulations, and can be dispensed such that different parts of the object are made, upon being hardened, of different cured modeling formulations, and hence are made of different hardened (e.g., cured) modeling materials or different mixtures or combinations of hardened (e.g., cured) modeling materials.

The final three-dimensional object is made of the modeling material or a combination of modeling materials or a combination of modeling material/s and support material/s or modification thereof (e.g., following curing). All these operations are well-known to those skilled in the art of solid freeform fabrication.

In some exemplary embodiments of the invention, an object is manufactured by dispensing a building material that comprises two or more different modeling material formulations, each modeling material formulation from a different dispensing head and/or nozzle of the inkjet printing apparatus. The modeling material formulations are optionally and preferably deposited in layers during the same pass of the printing heads. The modeling material formulations and/or combination of formulations within the layer are selected according to the desired properties of the object and according to the method parameters described herein.

An uncured building material can comprise one or more modeling material formulations, and can be dispensed such that different parts of the model object are made upon curing different modeling formulations, and hence are made of different cured modeling materials or different mixtures of cured modeling materials, or mixtures of cured modeling and support materials.

Herein throughout, the phrase “hardened support material” is also referred to herein interchangeably as “cured support material” or simply as “support material” and describes the part of the building material that is intended to support the fabricated final object during the fabrication process, and which is removed once the process is completed and a hardened modeling material is obtained.

Herein throughout, the phrase “support material formulation”, which is also referred to herein interchangeably as “support formulation”, describes a part of the uncured building material which is dispensed so as to form the support material, as described herein. The support material formulation is an uncured formulation. When a support material formulation is a curable formulation, it forms, upon exposure to a curing condition, a hardened support material.

Support materials, which can be either liquid materials or hardened, typically gel materials, are also referred to herein as sacrificial materials, which are removable after layers are dispensed and exposed to a curing energy, to thereby expose the shape of the final object.

Currently practiced support materials typically comprise a mixture of curable and non-curable materials.

Currently practiced support materials are typically water miscible, or water-dispersible or water-soluble.

Herein throughout, the term “water-miscible” describes a material which is at least partially dissolvable or dispersible in water, that is, at least 50% of the molecules move into the water upon mixing at room temperature, e.g., when mixed with water in equal volumes or weights, at room temperature. This term encompasses the terms “water-soluble” and “water dispersible”.

Herein throughout, the term “water-soluble” describes a material that when mixed with water in equal volumes or weights, at room temperature, a homogeneous solution is formed.

Herein throughout, the term “water-dispersible” describes a material that forms a homogeneous dispersion when mixed with water in equal volumes or weights, at room temperature.

The method and system of the present embodiments manufacture three-dimensional objects based on computer object data in a layer-wise manner by forming a plurality of layers in a configured pattern corresponding to the shape of the objects. The computer object data can be in any known format, including, without limitation, a Standard Tessellation Language (STL) or a StereoLithography Contour (SLC) format, Virtual Reality Modeling Language (VRML), Additive Manufacturing File (AMF) format, Drawing Exchange Format (DXF), Polygon File Format (PLY) or any other format suitable for Computer-Aided Design (CAD).

Each layer is formed by additive manufacturing apparatus which scans a two-dimensional surface and patterns it. While scanning, the apparatus visits a plurality of target locations on the two-dimensional layer or surface, and decides, for each target location or a group of target locations, whether or not the target location or group of target locations is to be occupied by building material formulation, and which type of building material formulation is to be delivered thereto. The decision is made according to a computer image of the surface.

In preferred embodiments of the present invention the AM comprises three-dimensional printing, more preferably three-dimensional inkjet printing. In these embodiments a building material formulation is dispensed from a dispensing head having a set of nozzles to deposit building material formulation in layers on a supporting structure. The AM apparatus thus dispenses building material formulation(s) in target locations which are to be occupied and leaves other target locations void. The apparatus typically includes a plurality of dispensing heads, each of which can be configured to dispense a different building material formulation. Thus, different target locations can be occupied by different building material formulations. The types of building material formulations can be categorized into two major categories: modeling material formulation and support material formulation. The support material formulation serves as a supporting matrix or construction for supporting the object or object parts during the fabrication process and/or other purposes, e.g., providing hollow or porous objects. Support constructions may additionally include modeling material formulation elements, e.g. for further support strength.

The final three-dimensional object is made of the modeling material or a combination of modeling materials or of modeling and support materials or modification thereof (e.g., following curing). All these operations are well-known to those skilled in the art of solid freeform fabrication.

In some exemplary embodiments of the invention an object is manufactured by dispensing one or more different modeling material formulations. When more than one modeling material formulation is used, each modeling material formulation is optionally and preferably dispensed from a different array of nozzles (belonging to the same or distinct dispensing heads) of the AM apparatus.

In some embodiments, the dispensing head of the AM apparatus is a multi-channel dispensing head, in which case different modeling material formulations can be dispensed from two or more arrays of nozzles that are located in the same multi-channels dispensing head. In some embodiments, arrays of nozzles that dispense different modeling material formulations are located in separate dispensing heads, for example, a first array of nozzles dispensing a first modeling material formulation is located in a first dispensing head, and a second array of nozzles dispensing a second modeling material formulation is located in a second dispensing head.

In some embodiments, an array of nozzles that dispense a modeling material formulation and an array of nozzles that dispense a support material formulation are both located in the same multi-channels dispensing head. In some embodiments, an array of nozzles that dispense a modeling material formulation and an array of nozzles that dispense a support material formulation are located in separate dispensing head heads.

The building material formulations are optionally and preferably deposited in layers during the same pass of the printing heads. The building material formulations and combination(s) of building material formulations within the layer are selected according to the desired properties of the object.

As discussed herein, the present inventors have designed and successfully prepared and practiced novel building material formulations that can be employed in additive manufacturing of a denture structure. The newly designed formulations are usable in additive manufacturing a monolithic denture structure that comprises a denture base and artificial teeth, preferably by three-dimensional inkjet printing.

Embodiments of the present invention relate to these newly designed modeling material formulations, to support material formulations that can be efficiently used in combination with these modeling material formulations, to additive manufacturing (e.g., three-dimensional inkjet printing) of a denture structure employing these formulations and to denture structures formed thereby.

Herein and in the art, the phrase “denture structure” describes a dental prosthesis intended to replace a missing tooth or teeth. Denture structures encompass an artificial tooth or teeth, and a base structure to support the artificial tooth or teeth. Denture structures can be partial dentures, typically comprised of a denture base and one tooth or several teeth, or complete dentures, typically comprised of a denture base and a plurality of teeth of the mandibular arch or the maxillary arch. Typically, a combination of complete dentures of both the mandibular arch and the maxillary arch are required. Denture structures are typically removable.

Embodiments of the present invention encompass additive manufacturing of artificial teeth, a denture base structure, each alone, and preferably, a monolithic structure of a denture base and artificial tooth or teeth. In some embodiments, the monolithic denture structure is a complete structure that comprises a base structure and a set of artificial teeth of the mandibular arch and/or the maxillary arch.

The manufacture of such a monolithic denture structure is enabled by the digital control of the color and mechanical properties of different parts of the structure, which in turn is enabled by the additive manufacturing method such as described herein and the respective newly designed curable formulations.

### Modeling Material Formulations:

According to an aspect of some embodiments of the present invention there is provided a modeling material formulation which usable in additive manufacturing of a denture structure as described herein.

According to some of any of the embodiments described herein, the modeling material formulation is such that is usable in three-dimensional inkjet printing and meets the process requirements of three-dimensional inkjet printing, as described herein.

According to some of any of the embodiments described herein, a modeling material formulation as described herein comprises one or more curable materials, and is also referred to herein as a curable formulation. A curable formulation is characterized in that its viscosity (e.g., at room temperature) increases, upon exposure to a curing condition as described herein, by at least 2-folds, preferably by at least 5-folds, and more preferably by at least one order of magnitude.

Herein throughout, a “curable material”, which is also referred to herein as a “solidifiable material” is a compound (e.g., monomeric or oligomeric or polymeric compound) which, when exposed to a curing condition (e.g., curing energy), as described herein, solidifies or hardens to form a cured modeling material as defined herein. Curable materials are typically polymerizable materials, which undergo polymerization and/or cross-linking when exposed to a suitable curing condition, typically a suitable energy source. A curable or solidifiable material is typically such that its viscosity increases by at least one order of magnitude when it is exposed to a curing condition.

In some of any of the embodiments described herein, a curable material can be a monomer, an oligomer or a short-chain polymer, each being polymerizable and/or cross-linkable as described herein.

In some of any of the embodiments described herein, when a curable material is exposed to a curing condition (e.g., curing energy such as, for example, radiation), it polymerizes by any one, or combination, of chain elongation and cross-linking.

In some of any of the embodiments described herein, a curable material is a monomer or a mixture of monomers which can form a polymeric modeling material upon a polymerization reaction, when exposed to a curing condition at which the polymerization reaction occurs. Such curable materials are also referred to herein as monomeric curable materials.

In some of any of the embodiments described herein, a curable material is an oligomer or a mixture of oligomers which can form a polymeric modeling material upon a polymerization reaction, when exposed to a curing condition at which the polymerization reaction occurs. Such curable materials are also referred to herein as oligomeric curable materials.

In some of any of the embodiments described herein, a curable material, whether monomeric or oligomeric, can be a mono-functional curable material or a multi-functional curable material.

Herein, a mono-functional curable material comprises one functional group that can undergo polymerization when exposed to a curing condition (e.g., curing energy).

A multi-functional curable material comprises two or more, e.g., 2, 3, 4 or more, functional groups that can undergo polymerization when exposed to a curing condition. Multi-functional curable materials can be, for example, di-functional, tri-functional or tetra-functional curable materials, which comprise 2, 3 or 4 groups that can undergo polymerization, respectively. The two or more functional groups in a multi-functional curable material are typically linked to one another by a linking moiety, as defined herein. When the linking moiety is an oligomeric moiety, the multi-functional group is an oligomeric multi-functional curable material.

Exemplary curable materials that are commonly used in additive manufacturing and in some of the present embodiments are acrylic materials.

Herein throughout, the term “acrylic materials” collectively encompasses materials bearing one or more acrylate, methacrylate, acrylamide and/or methacrylamide group(s).

The term “(meth)acrylate” and grammatical diversions thereof encompasses materials bearing one or more acrylate and/or methacrylate group(s).

The curable materials included in the formulations described herein may be defined by the properties of the materials before hardening, when appropriate. Such properties include, for example, molecular weight (MW), functionality (e.g., mono-functional or multi-functional), and viscosity

The curable materials included in the formulations described herein are otherwise defined by the properties provided by each material, when hardened. That is, the materials may be defined, when appropriate, by properties of a material formed upon exposure to a curing condition, for example, upon polymerization. These properties (e.g., Tg, HDT), are of a polymeric material formed upon curing any of the described curable materials alone.

As used herein, the term “curing” or “hardening” describes a process in which a formulation is hardened. This term encompasses polymerization of monomer(s) and/or oligomer(s) and/or cross-linking of polymeric chains (either of a polymer present before curing or of a polymeric material formed in a polymerization of the monomers or oligomers). The product of a curing reaction or of a hardening is therefore typically a polymeric material and in some cases a cross-linked polymeric material.

A “rate of hardening” as used herein represents the rate at which curing is effected, that is, the extent at which curable materials underwent polymerization and/or cross-linking in/within a given time period (for example, one minute). When a curable material is a polymerizable material, this phrase encompasses both a mol % of the curable materials in a formulation that underwent polymerization and/or cross-linking at the given time period, upon exposure to a curing condition; and/or the degree at which polymerization and/or cross-linking was effected, for example, the degree of chain elongation and/or cross-linking, at a given time period. Determining a rate of polymerization can be performed by methods known to those skilled in the art.

A “rate of hardening” can alternatively be represented by a degree at which a viscosity of a formulation charges at a given time period, that is, the rate at which the viscosity of a formulation increases upon exposure to curing condition.

Herein, the phrase “a condition that affects curing” or “a condition for inducing curing”, which is also referred to herein interchangeably as “curing condition” or “curing inducing condition” describes a condition which, when applied to a formulation that contains a curable material, induces at least partial polymerization of monomer(s) and/or oligomer(s) and/or cross-linking of polymeric chains. Such a condition can include, for example, application of a curing energy, as described hereinafter, to the curable material(s), and/or contacting the curable material(s) with chemically reactive components such as catalysts, co-catalysts, and activators.

When a condition that induces curing comprises application of a curing energy, the phrase “exposing to a curing condition” means that the dispensed layers, preferably each of the dispensed layers, is/are exposed to the curing energy and the exposure is typically performed by applying a curing energy to (e.g., each of) the dispensed layers.

A “curing energy” typically includes application of radiation or application of heat. The radiation can be electromagnetic radiation (e.g., ultraviolet or visible light), or electron beam radiation, or ultrasound radiation or microwave radiation, depending on the materials to be cured. The application of radiation (or irradiation) is effected by a suitable radiation source. For example, an ultraviolet or visible or infrared or Xenon lamp can be employed, as described herein.

A curable material, formulation or system that undergoes curing upon exposure to radiation is referred to herein interchangeably as “photopolymerizable” or “photoactivatable” or “photocurable”.

In some of any of the embodiments described herein, a curable material is a photopolymerizable material, which polymerizes or undergoes cross-linking upon exposure to radiation, as described herein, and in some embodiments the curable material is a UV-curable material, which polymerizes or undergoes cross-linking upon exposure to UV-vis radiation, as described herein.

In some embodiments, a curable material as described herein includes a polymerizable material that polymerizes via photo-induced radical polymerization.

According to some of any of the embodiments as described herein, all the curable materials in the formulation are photocurable materials, for example, (meth)acrylic materials.

According to some of any of the embodiments described herein, the modeling material formulation is such that when hardened it meets the requirements of common standards in the denture field, such as ISO 20795-1 Dentistry, ISO 10477 Dentistry and ISO 10993-1, as described herein.

According to some of any of the embodiments described herein, the modeling material formulation comprises two or more, three or more, four or more, five or more, or all, of the components described herein as Components A, B, C, D, E, F and G (see, Table 1 hereinbelow), and in some of these embodiments, it further comprises one or more of the components H, I, J, P and Dp (see, Table 1 hereinbelow).

According to some of any of the embodiments described herein, the modeling material formulation comprises two or more, three or more, four or more, five or more, and preferably all, of the following components:
a multi-functional (e.g., di-functional) urethane (meth)acrylate featuring, when hardened, high Tg (Component A);
a multi-functional (e.g., di-functional) non-aromatic (meth)acrylate featuring, when hardened, high Tg (Component B);
a filler in a form of micron-sized particles (Component C);
a multi-functional (e.g., di-functional) ethoxylated aromatic (meth)acrylate (Component D);
a mono-functional (meth)acrylate (Component E);
a multi-functional (e.g., tri-functional) (meth)acrylate (Component F); and
a multi-functional (e.g., di-functional) aliphatic urethane (meth)acrylate featuring, when hardened, low Tg (Component G).

According to some of any of the embodiments described herein, Component A is a multi-functional (e.g., di-functional) aliphatic urethane (meth)acrylate featuring, when hardened, Tg higher than 100° C.

According to some of any of the embodiments described herein, Component B is a multi-functional (e.g., di-functional) non-aromatic (meth)acrylate featuring, when hardened, Tg higher than 100° C.

According to some of any of the embodiments described herein, Component C comprises micron-sized filler particles functionalized by curable groups, as described herein.

According to some of any of the embodiments described herein, Component D is a multi-functional (e.g., di-functional) ethoxylated aromatic (meth)acrylate featuring less than 10 ethoxylated groups and/or featuring, when hardened, Tg that ranges from 50 to 150° C.

According to some of any of the embodiments described herein, Component E comprises at least one or at least two mono-functional (meth)acrylate(s).

According to some of any of the embodiments described herein, Component F is a multi-functional (e.g., tri-functional) cyclic (meth)acrylate.

According to some of any of the embodiments described herein, Component G is a multi-functional (e.g., di-functional) aliphatic urethane (meth)acrylate featuring, when hardened, Tg lower than 100° C.

According to some of any of the embodiments described herein, an amount of the filler (Component C) is no more than 20, or no more than 15, % by weight of the total weight of the formulation.

According to some of any of the embodiment as described herein, an amount of the Component D is no more than 20, or no more than 15, % by weight of the total weight of the formulation.

According to some of any of the embodiments described herein, an amount of the filler is no more than 20, or no more than 15, % by weight of the total weight of the formulation; and an amount of the Component D is no more than 20, or no more than 15, % by weight of the total weight of the formulation.

According to some of any of the embodiments described herein, the modeling material formulation comprises:
a multi-functional (e.g., di-functional) aliphatic urethane (meth)acrylate featuring, when hardened, Tg higher than 100° C. (Component A);
a multi-functional (e.g., di-functional) non-aromatic (meth)acrylate featuring, when hardened, Tg higher than 100° C. (Component B);
a filler in a form of micron-sized particles (Component C);
a multi-functional (e.g., di-functional) ethoxylated aromatic (meth)acrylate featuring less than 10 ethoxylated groups and/or featuring, when hardened, Tg that ranges from 50 to 150° C. (Component D);
a mono-functional (meth)acrylate (Component E);
a multi-functional (e.g., tri-functional) cyclic (meth)acrylate (Component F); and
a multi-functional (e.g., di-functional) aliphatic urethane (meth)acrylate featuring, when hardened, Tg lower than 100° C. (Component G),
wherein:
an amount of the filler is no more than 20, or no more than 15, % by weight of the total weight of the formulation; and
an amount of the Component D is no more than 20, or no more than 15, % by weight of the total weight of the formulation.

According to some of any of the embodiments described herein, the modeling material formulation comprises Component A as defined herein, Component B1 as defined herein, Component C as defined herein, Component D1 as defined herein, Components E1 and E2 as defined herein, Component F1 as defined herein, and Component G, as defined herein.

According to some of any of the embodiments described herein, an amount of Component A, as described herein in any of the respective embodiments and any combination thereof, ranges from 15 to 25, % by weight of the total weight of the formulation, including any intermediate values and subranges therebetween.

According to some of any of the embodiments described herein, an amount of each of Components B and C, as described herein in any of the respective embodiments and any combination thereof, is no more than 20, or no more than 15, % by weight of the total weight of the formulation, and, for example, ranges from about 5 to about 20, or preferably from about 5 to about 15, % by weight.

According to some of any of the embodiments described herein, an amount of Component D, as described herein in any of the respective embodiments and any combination thereof, is no more than 20, or no more than 15, % by weight of the total weight of the formulation, and preferably ranges from about 5 to about 20, or preferably from about 5 to about 15, % by weight, including any intermediate values and subranges therebetween.

According to some of any of the embodiments described herein, a total amount of Component E, as described herein in any of the respective embodiments and any combination thereof, ranges 30 to 40% by weight of the total weight of the formulation, including any intermediate values and subranges therebetween.

According to some of any of the embodiments described herein, an amount of Component F, as described herein in any of the respective embodiments and any combination thereof, ranges from 5 to 10, % by weight of the total weight of the formulation, including any intermediate values and subranges therebetween.

According to some of any of the embodiments described herein, an amount of Component G, as described herein in any of the respective embodiments and any combination thereof, ranges from about 5 to about 10, % by weight of the total weight of the formulation, including any intermediate values and subranges therebetween.

According to some of any of the embodiments described herein, the modeling material formulation comprises:
Component A, as described herein in any of the respective embodiments and any combination thereof, in an amount that ranges from 15 to 25, % by weight of the total weight of the formulation;
Components B and C, as described herein in any of the respective embodiments and any combination thereof, each independently in an amount of no more than 20, or no more than 15, % by weight of the total weight of the formulation;
Component D, as described herein in any of the respective embodiments and any combination thereof, in an amount of no more than 20, or no more than 15, % by weight of the total weight of the formulation;
Component E, as described herein in any of the respective embodiments and any combination thereof, in an amount of from 30 to 40% by weight of the total weight of the formulation;
Component F, as described herein in any of the respective embodiments and any combination thereof, in an amount of from 5 to 10, % by weight of the total weight of the formulation; and
Component G, as described herein in any of the respective embodiments and any combination thereof, in an amount of from 5 to 10, % by weight of the total weight of the formulation.

According to some of any of the embodiments described herein, the modeling material formulation comprises:
Component A, as described herein in any of the respective embodiments and any combination thereof, in an amount that ranges from 15 to 25, % by weight of the total weight of the formulation;
Component B, as described herein in any of the respective embodiments and any combination thereof, in an amount of no more than 20, or no more than 15, % by weight of the total weight of the formulation;
Component C, as described herein in any of the respective embodiments and any combination thereof, in an amount of no more than 20, or no more than 15, % by weight of the total weight of the formulation;
Component D, as described herein in any of the respective embodiments and any combination thereof, in an amount of no more than 20, or no more than 15, % by weight of the total weight of the formulation;
Components E1 and E2, as described herein in any of the respective embodiments and any combination thereof, in a total amount of from 30 to 40% by weight of the total weight of the formulation;
Component F1, as described herein in any of the respective embodiments and any combination thereof, in an amount of from 5 to 10, % by weight of the total weight of the formulation; and
Component G, as described herein in any of the respective embodiments and any combination thereof, in an amount of from 5 to 10, % by weight of the total weight of the formulation.

### Component A:

According to some of any of the embodiments described herein, Component A is a multi-functional (e.g., di-functional) urethane (meth)acrylate featuring, when hardened, Tg higher than 100° C.

According to some of any of the embodiments described herein, Component A is a multi-functional (e.g., di-functional) aliphatic urethane (meth)acrylate.

According to some of any of the embodiments described herein, Component A is a multi-functional (e.g., di-functional) aliphatic urethane (meth)acrylate featuring, when hardened, Tg higher than 100° C.

According to some of any of the embodiments described herein, Component A is a di-functional urethane (meth)acrylate featuring, when hardened, Tg higher than 100° C.

According to some of any of the embodiments described herein, Component A is a di-functional aliphatic urethane (meth)acrylate.

According to some of any of the embodiments described herein, Component A is a di-functional aliphatic urethane (meth)acrylate having Tg higher than 100° C.

According to some of any of the embodiments described herein, Component A is a di-functional urethane methacrylate featuring, when hardened, Tg higher than 100° C.

According to some of any of the embodiments described herein, Component A is a di-functional aliphatic urethane methacrylate.

According to some of any of the embodiments described herein, Component A is a di-functional aliphatic urethane methacrylate having Tg higher than 100° C.

According to some of any of the embodiments described herein, Component A features, when hardened, Tg that ranges from 100 to 200, or from 120 to 200, or from 100 to 150, or from 120 to 150,° C., including any intermediate values and subranges therebetween.

According to some of any of the embodiments described herein, an average molecular weight of Component A is lower than 1,000 grams/mol.

Any multi-functional aliphatic urethane (meth)acrylate is contemplated, and preferably such materials that are acceptable for inclusion in medical devices, such as devices for long term contact in a mucosal cavity and/or in edible (e.g., food-grade) products, and/or are characterized by a toxicity profile that is considered safe for long term contact with a mucosal cavity.

### Component B:

According to some of any of the embodiments described herein, Component B is a multi-functional (e.g., di-functional) non-aromatic (meth)acrylate featuring, when hardened, high Tg, for example, Tg higher than 100° C.

By “non-aromatic” it is meant a material that is devoid of aryl or heteroaryl groups or moieties, as these are defined herein.

Non-aromatic materials can be, for example, aliphatic or alicyclic.

According to some of any of the embodiments described herein, Component B is a multi-functional (e.g., di-functional) alicyclic (meth)acrylate featuring, when hardened, high Tg, for example, Tg higher than 100, and is referred to herein as Component B1.

According to some of any of the embodiments described herein, Component B1 is a di-functional alicyclic (meth)acrylate featuring, when hardened, high Tg, for example, Tg higher than 100° C.

According to some of any of the embodiments described herein, Component B1 is a di-functional alicyclic acrylate, or an alicyclic diacrylate, featuring, when hardened, high Tg, for example, Tg higher than 100° C.

According to some of any of the embodiments described herein, Component B1 comprises an alicyclic moiety of at least 6, 7, 8 or more carbon atoms.

According to some of any of the embodiments described herein, Component B1 comprises an alicyclic moiety which comprises 2, 3 or more fused rings.

According to some of any of the embodiments described herein, Component B or B1 features, when hardened, Tg that ranges from 100 to 300, or from 150 to 300, or from 100 to 200, or from 150 to 200,° C., including any intermediate values and subranges therebetween.

### Component C:

According to embodiments of the present invention, Component C is a filler in a particulate form, comprising a plurality of particles, preferably micron-sized particles.

The term “filler” as used herein describes an inert material that modifies the properties of a polymeric material and/or adjusts a quality of the end products.

Fillers (reinforcing materials) usable in additive manufacturing are typically inorganic particles of, for example, silica, calcium carbonate, clay, carbon black, and others.

In some of any of the embodiments described herein, the filler is or comprises silica particles.

In some embodiments, the average diameter of the particles is less than 1 micron, preferably less than 500 nm, preferably less than 200 nm and preferably less than 100 nm. In these embodiments, the silica particles are referred to also as silica nanoparticles.

In some embodiment, the average diameter of the particles ranges from 10 nm to 100 nm, or from 20 nm to 100 nm, or from 20 nm to 80 nm, or from 10 nm to 50 nm, including any intermediate values and subranges therebetween.

In some embodiments, at least a portion of such particles may aggregate, upon being introduced to the formulation. In some of these embodiments, the aggregate has an average size of no more than a few micrometers.

Any commercially available formulation of sub-micron silica particles is usable in the context of the present embodiments, including fumed silica, colloidal silica, precipitated silica, layered silica (e.g., montmorillonite), and aerosol assisted self-assembly of silica particles.

The silica particles can be such that feature a hydrophobic or hydrophilic surface. The hydrophobic or hydrophilic nature of the particles' surface is determined by the nature of the surface groups on the particles.

In a preferred embodiment, at least a portion, or all, of the silica particles are functionalized by curable functional groups (particles featuring curable groups on their surface).

The curable functional groups can be any polymerizable groups as described herein. In some embodiments, the curable functional groups are polymerizable by the same polymerization reaction as the curable monomers in the formulation, and/or when exposed to the same curing condition as the curable monomers. In some embodiments, the curable groups are (meth)acrylic (acrylic or methacrylic) groups, as defined herein, preferably (meth)acrylate groups.

By “at least a portion”, as used in the context of the present embodiments, it is meant at least 10%, or at least 20%, or at least 30%, or at least 40%, or at least 50%, or at least 60%, or at least 70%, or at least 80%, or at least 90%, or at least 95%, or at least 98%, of the particles.

In some embodiments, the silica particles are silica nanoparticles featuring acrylate and/or methacrylate groups on their surface.

According to some of any of the embodiments described herein, Component B, as described herein, preferable Component B1 as described herein, and Component C as described herein in any of the respective embodiments, are included in the formulation as a pre-mixed composition.

According to some of these embodiments, a weight ratio of Component B and Component C in the pre-mixed composition (and in a formulation comprising same) is about 1:1.

According to some of any of the embodiments described herein, a total amount of Component B (e.g., Component B1) and Component C ranges from about 15 to about 30, or from about 15 to about 25, or from about 2-to about 25, or from about 20 to about 30, % by weight of the total weight of the formulation, including any intermediate values and subranges therebetween.

### Component D:

According to some of any of the embodiments described herein, Component D is a multi-functional ethoxylated (meth)acrylate.

According to some of any of the embodiments described herein, Component D is multi-functional (e.g., di-functional) ethoxylated aromatic (meth)acrylate, which comprises one or more aromatic (aryl or heteroaryl) moieties.

According to some of any of the embodiments described herein, Component D comprises a Bisphenol A moiety as a branching unit from which two or three ethoxylated moieties that terminate by (meth)acrylate groups extend.

According to some of any of the embodiments described herein, Component D is a di-functional ethoxylated aromatic (meth)acrylate.

According to some of any of the embodiments described herein, Component D is multi-functional (e.g., di-functional) ethoxylated aromatic (meth)acrylate featuring, when hardened, Tg lower than 200° C.

According to some of any of the embodiments described herein, Component D is multi-functional (e.g., di-functional) ethoxylated aromatic (meth)acrylate featuring, when hardened, Tg that ranges from 50 to 150° C., including any intermediate values and subranges therebetween.

According to some of any of the embodiments described herein, Component D is a di-functional ethoxylated aromatic (meth)acrylate featuring, when hardened, Tg that ranges from 50 to 150° C., including any intermediate values and subranges therebetween.

According to some of any of the embodiments described herein, Component D is a multi-functional (e.g., di-functional) ethoxylated aromatic methacrylate featuring, when hardened, Tg that ranges from 50 to 150° C., including any intermediate values and subranges therebetween.

According to some of any of the embodiments described herein, Component D is a di-functional ethoxylated aromatic methacrylate (ethoxylated aromatic dimethacrylate) featuring, when hardened, Tg that ranges from 50 to 150° C., including any intermediate values and subranges therebetween.

According to some of any of the embodiments described herein, Component D comprises less than 10 ethoxylated moieties, or less than 8, or less than 6 or less than 5, ethoxylated moieties.

According to some of any of the embodiments described herein, Component D comprises a total of 4 ethoxylated moieties.

According to some of any of the embodiments described herein, Component D is a multi-functional (e.g., di-functional) ethoxylated aromatic (meth)acrylate featuring, when hardened, Tg that ranges from 50 to 150° C., including any intermediate values and subranges therebetween and/or comprising less than 10 ethoxylated moieties, or less than 8, or less than 6 or less than 5, ethoxylated moieties, for example, 4 ethoxylated moieties. Such a component is referred to herein as Component D1.

According to some of any of the embodiments described herein, Component D1 is a multi-functional (e.g., di-functional) ethoxylated aromatic (meth)acrylate featuring, when hardened, Tg that ranges from 50 to 150° C., including any intermediate values and subranges therebetween and comprises less than 10 ethoxylated moieties, or less than 8, or less than 6 or less than 5, ethoxylated moieties, for example, 4 ethoxylated moieties.

According to some of any of the embodiments described herein, Component D1 is a di-functional ethoxylated aromatic (meth)acrylate featuring, when hardened, Tg that ranges from 50 to 150° C., including any intermediate values and subranges therebetween and comprises less than 10 ethoxylated moieties, or less than 8, or less than 6 or less than 5, ethoxylated moieties, for example, 4 ethoxylated moieties.

According to some of any of the embodiments described herein, Component D1 is a multi-functional (e.g., di-functional) ethoxylated aromatic methacrylate featuring, when hardened, Tg that ranges from 50 to 150° C., including any intermediate values and subranges therebetween and comprises less than 10 ethoxylated moieties, or less than 8, or less than 6 or less than 5, ethoxylated moieties, for example, 4 ethoxylated moieties.

According to some of any of the embodiments described herein, Component D1 is a di-functional ethoxylated aromatic methacrylate featuring, when hardened, Tg that ranges from 50 to 150° C., including any intermediate values and subranges therebetween and comprises less than 10 ethoxylated moieties, or less than 8, or less than 6 or less than 5, ethoxylated moieties, for example, 4 ethoxylated moieties.

### Component E:

According to some of any of the embodiments described herein, Component E comprises one or more mono-functional (meth)acrylate materials.

According to some of any of the embodiments described herein, Component E comprises two or more mono-functional (meth)acrylate materials.

According to some of any of the embodiments described herein, Component E comprises two or more mono-functional (meth)acrylate materials, at least one is a mono-functional acrylate, also referred to herein as Component E1 and at least one is a mono-functional methacrylate, also referred to herein as Component E2.

According to some of any of the embodiments described herein, each of the mono-functional materials has an average molecular weight lower than 1,000 grams/mol or lower than 500 grams/mol.

According to some of any of the embodiments described herein, at least one of Components E1 and E2, or both, is a hydrophilic and/or an amphiphilic material.

As used herein throughout, the term “hydrophilic” describes a physical property of a material or a portion of a material (e.g., a chemical group in a compound) which accounts for transient formation of bond(s) with water molecules, typically through hydrogen bonding.

Hydrophilic materials dissolve more readily in water than in oil or other hydrophobic solvents. Hydrophilic materials can be determined by, for example, as having Log P lower than 0.5, when Log P is determined in octanol and water phases.

Hydrophilic materials can alternatively, or in addition, be determined as featuring a lipophilicity/hydrophilicity balance (HLB), according to the Davies method, of at least 10, or of at least 12.

As used herein throughout, the term “amphiphilic” describes a property of a material that combines both hydrophilicity, as described herein for hydrophilic materials, and hydrophobicity or lipophilicity, as defined herein for hydrophobic materials.

Amphiphilic materials typically comprise both hydrophilic groups as defined herein and hydrophobic groups, as defined herein, and are substantially soluble in both water and a water-immiscible solvent (oil).

Amphiphilic materials can be determined by, for example, as having Log P of 0.8 to 1.2, or of about 1, when Log P is determined in octanol and water phases.

Amphiphilic materials can alternatively, or in addition, be determined as featuring a lipophilicity/hydrophilicity balance (HLB), according to the Davies method, of 3 to 12, or 3 to 9.

A hydrophilic material or portion of a material (e.g., a chemical group in a compound) is one that is typically charge-polarized and capable of hydrogen bonding.

Amphiphilic materials typically comprise one or more hydrophilic groups (e.g., a charge-polarized group), in addition to hydrophobic groups.

Hydrophilic materials or groups, and amphiphilic materials, typically include one or more electron-donating heteroatoms which form strong hydrogen bonds with water molecules. Such heteroatoms include, but are not limited to, oxygen and nitrogen. Preferably, a ratio of the number of carbon atoms to a number of heteroatoms in a hydrophilic materials or groups is 10:1 or lower, and can be, for example, 8:1, more preferably 7:1, 6:1, 5:1 or 4:1, or lower. It is to be noted that hydrophilicity and amphiphilicity of materials and groups may result also from a ratio between hydrophobic and hydrophilic moieties in the material or chemical group, and does not depend solely on the above-indicated ratio.

A hydrophilic or amphiphilic material can have one or more hydrophilic groups or moieties. Hydrophilic groups are typically polar groups, comprising one or more electron-donating heteroatoms such as oxygen and nitrogen.

Exemplary hydrophilic groups include, but are not limited to, an electron-donating heteroatom, a carboxylate, a thiocarboxylate, oxo (═O), a linear amide, hydroxy, a (C1-4)alkoxy, an (C1-4)alcohol, a heteroalicyclic (e.g., having a ratio of carbon atoms to heteroatoms as defined herein), a cyclic carboxylate such as lactone, a cyclic amide such as lactam, a carbamate, a thiocarbamate, a cyanurate, an isocyanurate, a thiocyanurate, urea, thiourea, an alkylene glycol (e.g., ethylene glycol or propylene glycol), and a hydrophilic polymeric or oligomeric moiety, as these terms are defined hereinunder, and any combinations thereof (e.g., a hydrophilic group that comprises two or more of the indicated hydrophilic groups).

In some embodiments, the hydrophilic group is, or comprises, an electron donating heteroatom, a carboxylate, a heteroalicyclic, an alkylene glycol and/or a hydrophilic oligomeric moiety.

An amphiphilic moiety or group typically comprises one or more hydrophilic groups as described herein and one or more hydrophobic groups, or, can a heteroatom-containing group or moiety in which the ratio of number of carbon atoms to the number of heteroatoms accounts for amphiphilicity.

A hydrophilic or amphiphilic mono-functional curable material according to some embodiments of the present invention can be a hydrophilic acrylate represented by Formula A1:

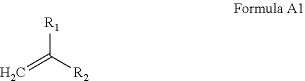

wherein R₁ and R₂ are as defined herein and at least one of R₁ and R₂ is and/or comprises a hydrophilic or amphiphilic moiety or group, as defined herein.

In some of any of these embodiments, the carboxylate group, —C(═O)—ORa, comprises Ra which is a hydrophilic or amphiphilic moiety or group, as defined herein. Exemplary Ra groups in the context of these embodiments include, but are not limited to, heteroalicyclic groups (having a ratio of 10:1 or 8:1 or 6:1 or 5:1 or lower of carbon atoms to electron-donating heteroatoms, such as morpholine, tetrahydrofurane, oxalidine, and the likes), hydroxyl, C(1-4)alkoxy, thiol, alkylene glycol or a hydrophilic or amphiphilic polymeric or oligomeric moiety, as described herein. An exemplary hydrophilic monomeric mono-functional acrylate is acryloyl morpholine (ACMO).

Exemplary hydrophilic or amphiphilic oligomeric mono-functional curable materials include, but are not limited to, a mono-(meth)acrylated urethane oligomer derivative of polyethylene glycol, a mono-(meth)acrylated polyol oligomer, a mono-(meth)acrylated oligomer having hydrophilic substituents, a mono-(meth)acrylated polyethylene glycol (e.g., methoxypolyethylene glycol), and a mono urethane acrylate.

According to some of any of the embodiments described herein, Component E1 is a hydrophilic or amphiphilic mono-functional methacrylate.

According to some of any of the embodiments described herein, Component E2 is a mono-functional acrylate, and in some embodiments, it is a mono-functional acrylate that has an alicyclic group as Ra in Formula A1.

According to some of any of the embodiments described herein, Component E1 is a hydrophilic or amphiphilic mono-functional methacrylate and Component E2 is a mono-functional acrylate, and in some embodiments, it is a mono-functional acrylate that has an alicyclic group as Ra in Formula A1.

According to some of any of the embodiments described herein, a weight ratio of the mono-functional methacrylate (E1) and the mono-functional acrylate (E2) ranges from 2:1 to 1:2, or is about 1:1.

According to some of any of the embodiments described herein, an amount of each of the mono-functional acrylate (E2) and the mono-functional methacrylate (E1) independently ranges from 10 to 20, or from 15 to 20, % by weight of the total weight of the formulation.

According to some of any of the embodiments described herein, a total amount of the one or more mono-functional (meth)acrylate(s) (e.g., E1 and E2) ranges from 30 to 40% by weight of the total weight of the formulation, including any intermediate values and subranges therebetween.

According to some of any of the embodiments described herein, at least one or both of the mono-functional acrylate (E2) and the mono-functional methacrylate (E1) features, when hardened, Tg lower than 100° C. or lower than 80° C.

### Component F:

According to some of any of the embodiments describes herein, Component F is a tri-functional (meth)acrylate.

According to some of any of the embodiments describes herein, Component F is a multi-functional (e.g., tri-functional) (meth)acrylate that features, when hardened, Tg higher than 150, or higher than 180, or higher than 200,° C.

According to some of any of the embodiments describes herein, Component F is a multi-functional (e.g., tri-functional) cyclic (meth)acrylate, which comprises one or more cyclic moieties such as aryl and/or alicyclic, and is also referred to herein as Component F1.

According to some of any of the embodiments describes herein, Component F1 is a tri-functional cyclic (meth)acrylate, which comprises one or more cyclic moieties such as aryl and/or alicyclic.

According to some of any of the embodiments describes herein, Component F1 is a tri-functional cyclic methacrylate, or cyclic trimethacrylate, which comprises one or more cyclic moieties such as aryl and/or alicyclic.

According to some of any of the embodiments described herein, Component F or F1 features, when hardened, high Tg, for example, Tg higher than 100, or higher than 150, or higher than 200, or even higher than 250,° C.

According to some of any of the embodiments describes herein, Component F1 is a tri-functional cyclic methacrylate, or cyclic trimethacrylate, which comprises one or more cyclic moieties such as aryl and/or alicyclic, and features, when hardened, high Tg, for example, Tg higher than 100, or higher than 150, or higher than 200, or even higher than 250,° C.

In some of any of the embodiments of Component For F1, the cyclic moiety is a branching unit as defined herein.

In some of any of the embodiments of Component For F1, the cyclic moiety is or comprises a cyanurate or an isocyanurate.

In some of any of the embodiments of Component For F1, the cyclic moiety is or comprises a cyanurate or an isocyanurate and is a branching unit, from which moieties that comprise the (meth)acrylate groups extend.

### Component G:

According to some of any of the embodiments described herein, Component G is a multi-functional (e.g., di-functional) aliphatic urethane (meth)acrylate, having an average MW of at least 1,000 grams/mol. Such a component is also referred to herein as an oligomeric curable material. According to some of any of the embodiments described herein, Component G is a di-functional aliphatic urethane (meth)acrylate, having an average MW of at least 1,000 grams/mol.

According to some of any of the embodiments described herein, Component G is a multi-functional (e.g., di-functional) aliphatic urethane methacrylate, having an average MW of at least 1,000 grams/mol.

According to some of any of the embodiments described herein, Component G is a di-functional aliphatic urethane methacrylate, having an average MW of at least 1,000 grams/mol.

According to some of any of the embodiments described herein, Component G features, when hardened, low Tg.

According to some of any of the embodiments described herein, Component G features, when hardened, Tg lower than 100° C. or lower than 80° C.

According to some of any of the embodiments described herein, Component G is a non-polar (e.g., non-hydrophilic or hydrophobic) multi-functional (e.g., di-functional) aliphatic urethane (meth)acrylate as described herein.

### Exemplary Formulations

According to exemplary embodiments, the modeling material formulation comprises:
Component A as described herein in any of the respective embodiments and any combination thereof in an amount that ranges from 15 to 25, % by weight of the total weight of the formulation;
Component B as described herein in any of the respective embodiments and any combination thereof, preferably Component B1, in an amount of no more than 20, or no more than 15, % by weight of the total weight of the formulation;
Component C as described herein in any of the respective embodiments and any combination thereof, in an amount of no more than 20, or no more than 15, % by weight of the total weight of the formulation;
Component D as described herein in any of the respective embodiments and any combination thereof, preferably Component D1, in an amount of no more than 20, or no more than 15, % by weight of the total weight of the formulation;
Component E as described herein in any of the respective embodiments and any combination thereof, preferably a mixture of Components E1 and E2, in a total amount of from 30 to 40% by weight of the total weight of the formulation;
Component F as described herein in any of the respective embodiments and any combination thereof, preferably Component F1, in an amount of from 5 to 10, % by weight of the total weight of the formulation; and
Component G as described herein in any of the respective embodiments and any combination thereof, in an amount of from 5 to 10, % by weight of the total weight of the formulation.

According to exemplary embodiments, the modeling material formulation comprises:
as Component A-a di-functional aliphatic urethane methacrylate featuring, when hardened, Tg higher than 100° C.;
as Component B-Component B1 which is a di-functional alicyclic acrylate featuring, when hardened, Tg higher than 100° C.;
as Component C comprises silica particles having curable groups attached thereto;
as Component D-Component D1 which is a di-functional ethoxylated aromatic methacrylate featuring less than 5 ethoxylated groups and, when hardened, Tg that ranges from 50 to 150° C.;
as Component E a mono-functional acrylate (E2) and a mono-functional methacrylate (E1), each independently in an amount of from 10 to 20, or from 15 to 20, % by weight, of the total weight of the formulation;
as Component F-Component F1 which is a tri-functional isocyanurate triacrylate; and
as Component G a di-functional aliphatic urethane dimethacrylate featuring, when hardened, Tg lower than 100° C. and an average MW of at least 1,000 grams/mol.

According to exemplary embodiments, the modeling material formulation comprises:
as Component A—a di-functional aliphatic urethane methacrylate featuring, when hardened, Tg higher than 100° C., in an amount that ranges from 15 to 25, % by weight of the total weight of the formulation;
as Component B—Component B1 which is a di-functional alicyclic acrylate featuring, when hardened, Tg higher than 100° C., in an amount of no more than 20, or no more than 15, % by weight of the total weight of the formulation;
as Component C comprises silica particles having curable groups attached thereto, in an amount of no more than 20, or no more than 15, % by weight of the total weight of the formulation;
as Component D—Component D1 which is a di-functional ethoxylated aromatic methacrylate featuring less than 5 ethoxylated groups and, when hardened, Tg that ranges from 50 to 150° C., in an amount of no more than 20, or no more than 15, % by weight of the total weight of the formulation;
as Component E a mono-functional acrylate (E2) and a mono-functional methacrylate (E1), each independently in an amount of from 10 to 20, or from 15 to 20, % by weight, of the total weight of the formulation, in a total amount of from 30 to 40% by weight of the total weight of the formulation;
as Component F—Component F1 which is a tri-functional isocyanurate triacrylate, in an amount of from 5 to 10, % by weight of the total weight of the formulation; and
as Component G a di-functional aliphatic urethane dimethacrylate featuring, when hardened, Tg lower than 100° C. and an average MW of at least 1,000 grams/mol, in an amount of from 5 to 10, % by weight of the total weight of the formulation.

According to some of any of the embodiments described herein, the modeling material formulation is devoid of methyl methacrylate and/or methylacrylate, and/or is such that the hardened modeling material is devoid of poly (methyl methacrylate) (PMMA).

Herein throughout, by “devoid of” it is meant less than 1%, or less than 0.1%, or less than 0.01%, or less than 0.001%, or null.

### Additional Components:

According to some of any of the embodiments described herein, the modeling material formulation further comprises a dispersant (Component H).

According to some of these embodiments, the dispersant features curable groups, preferably (meth)acrylic groups.

According to some of any of the embodiments described herein, the dispersant is a multi-functional (e.g., di-functional) aliphatic silicon (meth)acrylate.

According to some of any of the embodiments described herein, the dispersant is a di-functional aliphatic silicon (meth)acrylate.

According to some of any of the embodiments described herein, the dispersant is a multi-functional (e.g., di-functional) aliphatic silicon acrylate.

According to some of any of the embodiments described herein, the dispersant is a di-functional aliphatic silicon acrylate.

According to some of any of the embodiments described herein, the dispersant has an average MW of at least 1,000, or at least 2,000, or at least 3,000 grams/mol, and is considered as an oligomeric material.

According to some of any of the embodiments described herein, the dispersant is a multi-functional (e.g., di-functional) aliphatic silicon (meth)acrylate, having an average MW of at least 1,000 grams/mol as described herein.

According to some of any of the embodiments described herein, the dispersant is a di-functional aliphatic silicon (meth)acrylate, having an average MW of at least 1,000 grams/mol as described herein.

According to some of any of the embodiments described herein, the dispersant is a multi-functional (e.g., di-functional) aliphatic silicon acrylate, having an average MW of at least 1,000 grams/mol as described herein.

According to some of any of the embodiments described herein, the dispersant is a di-functional aliphatic silicon acrylate, having an average MW of at least 1,000 grams/mol as described herein.

According to some of any of the embodiments described herein, the dispersant features, when hardened, low Tg, preferably lower than 0, or lower than −20, or lower than −50,° C.

According to some of any of the embodiments described herein, an amount of the dispersant ranges from 0.1 to 0.5, % by weight, of the total weight of the formulation, including any intermediate values and subranges therebetween.

According to some of any of the embodiments described herein, the modeling material formulation further comprises an inhibitor (Component I), as described herein, for example, a phenol-type inhibitor or any other inhibitor that is commonly used in medical devices or applications and/in food products.

According to some of any of the embodiments described herein, an amount of the inhibitor ranges from 0.001 to 0.010, % by weight, of the total weight of the formulation, including any intermediate values and subranges therebetween.

According to some of any of the embodiments described herein, the modeling material formulation further comprises at least one photoinitiator (Component J).

According to some of any of the embodiments described herein, an amount of the photoinitiator ranges from 1 to 5, % by weight of the total weight of the formulation, including any intermediate values and subranges therebetween.

According to some of any of the embodiments described herein, the photoinitiator(s) comprises, or consists essentially of, a phosphine oxide-type (e.g., mono-acrylated (MAPO) or bis-acrylated phosphine oxide-type (BAPO) photoinitiator.

Exemplary monoacyl and bisacyl phosphine oxides include, but are not limited to, 2,4,6-trimethylbenzoyldiphenyl phosphine oxide, bis(2,4,6-trimethylbenzoyl)phenylphosphine oxide, dibenzoylphenylphosphine oxide, bis(2,6-dimethoxybenzoyl)phenyl phosphine oxide, tris(2,4-dimethylbenzoyl) phosphine oxide, tris(2-methoxybenzoyl)phosphine oxide, 2,6-dimethoxybenzoyldiphenyl phosphine oxide, 2,6-dichlorobenzoyldiphenyl phosphine oxide, 2,3,5,6-tetramethylbenzoyldiphenyl phosphine oxide, benzoyl-bis(2,6-dimethylphenyl) phosphonate, and 2,4,6-trimethylbenzoylethoxyphenyl phosphine oxide. Commercially available phosphine oxide photoinitiators capable of free-radical initiation when irradiated at wavelength ranges of greater than about 380 nm to about 450 nm include 2,4,6-trimethylbenzoyldiphenyl phosphine oxide (TPO), bis(2,4,6-trimethylbenzoyl) phenyl phosphine oxide (marketed as IRGACURE® 819), bis(2,6-dimethoxybenzoyl)-(2,4,4-trimethylpentyl) phosphine oxide (marketed as CGI 403), a 25:75 mixture, by weight, of bis(2,6-dimethoxybenzoyl)-2,4,4-trimethylpentyl phosphine oxide and 2-hydroxy-2-methyl-1-phenylpropan-1-one (marketed as IRGACURE® 1700), a 1:1 mixture, by weight, of bis(2,4,6-trimethylbenzoyl) phenyl phosphine oxide and 2-hydroxy-2-methyl-1-phenylpropane-1-one (marketed as DAROCUR® 4265), and ethyl 2,4,6-trimethylbenzylphenyl phosphinate (LUCIRIN LR8893X).

In an exemplary embodiments, the photoinitiator is or comprises bis(2,4,6-trimethylbenzoyl) phenyl phosphine oxide (marketed as IRGACURE® 819).

In an exemplary embodiments, the photoinitiator is devoid of 2,4,6-trimethylbenzoyldiphenyl phosphine oxide (marketed as TPO) and/or bis(2,4,6-trimethylbenzoyl) phenyl phosphine oxide (marketed as IRGACURE® 819).

According to some of any of the embodiments described herein, the modeling material formulation is a clear (e.g., transparent), colorless formulation, which is devoid of a coloring agent.

According to some of any of the embodiments described herein, the modeling material formulation further comprises one or more coloring agent(s) (Component P).

The coloring agent can be a pigment or a dye and is preferably a pigment.

The pigments can be organic and/or inorganic and/or metallic pigments, and in some embodiments the pigments are nanoscale pigments, which include nanoparticles.

Exemplary inorganic pigments include nanoparticles of titanium oxide, and/or of zinc oxide and/or of silica. Exemplary organic pigments include nano-sized carbon black.

In some embodiments, combinations of white and color pigments are used to prepare colored cured materials.

According to some of any of the embodiments described herein, the coloring agent comprises a mixture of a pigment and at least one (meth)acrylic material, such that the pigment is introduced to the formulation within this mixture.

According to some of any of the embodiments described herein, the pigment is a white pigment and the formulation provides a white hardened material.

According to some of any of the embodiments described herein, the coloring agent comprises a mixture of a white pigment and one or more curable materials such as (meth)acrylic materials, such that the pigment is introduced to the formulation within this mixture.

According to some of these embodiments, an amount of the white pigment in the mixture ranges from 20 to 50% by weight of the total weight of the mixture, including any intermediate values and subranges therebetween.

According to some of these embodiments, an amount of the coloring agent, which is a mixture of a white pigment and at least one (meth)acrylic material ranges from 1 to 5% by weight of the total weight of the formulation.

According to some of any of the embodiments described herein, the pigment is a cyan pigment and the formulation provides a cyan hardened material.

According to some of any of the embodiments described herein, the coloring agent comprises a mixture of a cyan pigment and one or more curable materials such as (meth)acrylic materials, such that the cyan pigment is introduced to the formulation within this mixture.

According to some of these embodiments, an amount of the cyan pigment in the mixture ranges from 0.01 to 1, or from 0.05 to 0.5, or from 0.1 to 0.2, % by weight of the total weight of the mixture.

According to some of these embodiments, an amount of the coloring agent, which is a mixture of a cyan pigment and at least one (meth)acrylic material ranges from 0.1 to 1% by weight of the total weight of the formulation.

According to some of any of the embodiments described herein, the pigment is a yellow pigment and the formulation provides a yellow hardened material.

According to some of any of the embodiments described herein, the coloring agent comprises a mixture of a yellow pigment and one or more curable materials such as (meth)acrylic materials, such that the yellow pigment is introduced to the formulation within this mixture.

According to some of these embodiments, an amount of the yellow pigment in the mixture ranges from 0.01 to 1, or from 0.05 to 0.5, or from 0.1 to 0.2, % by weight of the total weight of the mixture.

According to some of these embodiments, an amount of the coloring agent, which is a mixture of a yellow pigment and at least one (meth)acrylic material ranges from 0.1 to 1% by weight of the total weight of the formulation.

According to some of any of the embodiments described herein, the pigment is a magenta pigment and the formulation provides a magenta hardened material.

According to some of any of the embodiments described herein, the coloring agent comprises a mixture of a magenta pigment and one or more curable materials such as (meth)acrylic materials, such that the magenta pigment is introduced to the formulation within this mixture.

According to some of these embodiments, an amount of the magenta pigment in the mixture ranges from 0.01 to 1, or from 0.05 to 0.5, or from 0.1 to 0.2, % by weight of the total weight of the mixture.

According to some of these embodiments, an amount of the coloring agent, which is a mixture of a magenta pigment and at least one (meth)acrylic material ranges from 0.1 to 1% by weight of the total weight of the formulation.

According to some of any of the embodiments described herein, the formulation comprises one or more of a white, magenta, cyan, and yellow coloring agents, and in some of these embodiments, each pigment is introduced to the formulation in a mixture with curable materials as described herein.

According to some of any of the embodiments described herein, the coloring agent further comprises a pigment dispersant (Component Dp). Preferred pigment dispersants are such that has a plurality of groups that feature an affinity to the pigment.

According to some of any of the embodiments described herein, the modeling material formulation comprises Components H, I, and J, as described herein in any of the respective embodiments. An exemplary such a formulation is a clear colorless formulation, that is devoid of a coloring agent.

According to some of any of the embodiments described herein, the modeling material formulation comprises Components H, I, J and P, as described herein in any of the respective embodiments. An exemplary such a formulation is a white formulation that comprises a white pigment as described herein.

According to some of any of the embodiments described herein, the modeling material formulation comprises Components H, I, J, P and Dp, as described herein in any of the respective embodiments. Exemplary such formulations are the cyan, magenta and yellow formulation as described herein.

### Kits:

In some of any of the embodiments described herein there is provided a kit comprising one or more modeling material formulations as described herein in any of the respective embodiments and any combination thereof.

In some of any of the embodiments described herein there is provided a kit comprising two or more modeling material formulations as described herein in any of the respective embodiments and any combination thereof. In some of these embodiments, each formulation is packaged individually in the kit.

In exemplary embodiments, the kit comprises a combination of two or more formulations that differ from one another by the presence and/or type of the coloring agent or pigment.

In exemplary embodiments, the kit comprises two or more of a clear, white, cyan, magenta, and yellow formulations as described herein in any of the respective embodiments.

A kit as described herein is usable for additive manufacturing of a denture structure as described herein, particularly a monolithic denture structure as described herein.

In exemplary embodiments, the formulations are packaged within the kit in a suitable packaging material, preferably, an impermeable material (e.g., water- and gas-impermeable material), and further preferably an opaque material. In some embodiments, the kit further comprises instructions to use the formulations in an additive manufacturing process, preferably a 3D inkjet printing process as described herein. The kit may further comprise instructions to use the formulations in the process in accordance with the method as described herein.

According to some of any of the embodiments that relate to a kit, the kit may further comprise a support material formulation. Any support material formulation that is usable, for example, in AM such as 3D inkjet printing is contemplated.

According to some of these embodiments, the support material formulation is as described herein in any of the respective embodiments.

### Support Material Formulation:

The present inventors have designed and successfully practiced a support material formulation that is suitable for use with the modeling material formulations of the present embodiments.

According to some embodiments of this aspect of the present invention, the support material formulation comprises:
a non-curable water-soluble or water-miscible polymeric material, in an amount of from about 40 to about 60% by weight of the total weight of the formulation;
a hydrophilic mono-functional (meth)acrylate, in an amount of from 15 to 25% by weight of the total weight of the formulation;
a hydrophilic mono-functional (meth)acrylamide in an amount of from 10 to 20% by weight of the total weight of the formulation; and
a multi-functional non-aromatic (e.g., aliphatic or alicyclic) (meth)acrylate in an amount of from 1 to 5% by weight of the total weight of the formulation.

According to some of any of these embodiments, the non-curable polymeric material comprises a polyol.

Herein and in the art, the term “polyol” describes a polymeric material that features two or more free hydroxy groups, typically from about 10 to dozens or hundreds free hydroxy groups. Representative examples of a polyol include, without limitation, a polyester polyol, a polyether polyol and a urethane polyol. Preferably, the polyol is a polyether polyol such as, for example, a poly (alkylene glycol).

The polyol can be a linear polyol or a non-linear (e.g., branched polyol).

According to some of any of the embodiments described herein, the polyol is a poly (alkylene glycol), for example, a poly (ethylene glycol) or a poly (propylene glycol) or a mixture thereof. In some embodiments, the polyol is or comprises a poly (propylene glycol).

According to some of any of the embodiments described herein, the polyol comprises an alkoxylated branched polyol, such as, for example, marketed as Polyol 3165.

According to some of any of the embodiments described herein, the polyol has an average molecular weight lower than 1,200, or lower than 1,000 grams/mol.

The polyol can have an average molecular weight that ranges from about 200 to about 1,100, or from about 400 to about 1,100, including any intermediate values and subranges therebetween.

According to some of any of the embodiments described herein, the support material formulation further comprises a non-curable water-soluble or water-miscible non-polymeric material, such as a diol, or triol, or glycerol, etc. In exemplary embodiments, it a diol such as propanediol.

According to some of any of the embodiments described herein, the support material formulation comprises a mixture of polymeric and non-polymeric materials as described herein, and in some of these embodiments it comprises a mixture of a poly (alkylene glycol), a branched polyol, and a diol. In some embodiments, the total amount of these materials ranges from about 40 to about 80 5 by weight of the total weight of the formulation.

According to some of any of the embodiments described herein, the formulation comprises one or more mono-functional curable materials.

According to some of any of the embodiments described herein, one or more, or each, of the mono-functional curable material(s) is a hydrophilic material, as defined herein, for example, having Formula A1.

According to some embodiments, at least one of the mono-functional curable materials is a mono-functional (meth)acrylate, preferably a mono-functional acrylate having Formula A1 in which R₁ is a carboxylate. In some of these embodiments, R′ is a poly (alkylene glycol), as defined herein. An exemplary such hydrophilic mono-functional acrylate is hexa (ethylene glycol) acrylate, (6-PEA).

According to some embodiments, at least one of the mono-functional curable materials is a mono-functional (meth)acrylamide, preferably a mono-functional acrylate having Formula A1 in which R₁ is amide. According to some embodiments, at least one of the mono-functional curable materials is a mono-functional acrylamide. In some of these embodiments, Ra is a shorty alkyl, of 2 to 8, or 2 to 6, or 2 to 4, carbon atom in length, which terminates by a hydrophilic group as defined herein. An exemplary such hydrophilic group is hydroxy alkyl, for example, hydroxyethyl.

According to some of any of the embodiments described herein, one or more, or each, of the mono-functional curable material(s) is a water-miscible or water-soluble material, as defined herein.

According to some of any of the embodiments described herein, the formulation comprises a multi-functional (e.g., di-functional) aliphatic or alicyclic (meth)acrylate.

According to some embodiments, the formulation comprises a di-functional aliphatic or alicyclic (meth)acrylate.

According to some of any of the embodiments described herein, the formulation comprises a multi-functional (e.g., di-functional) aliphatic or alicyclic acrylate.

According to some of any of the embodiments described herein, the formulation comprises a di-functional aliphatic or alicyclic acrylate, that is, an aliphatic or alicyclic diacrylate.

According to some of any of the embodiments described herein, the formulation comprises a multi-functional (e.g., di-functional) alicyclic (meth)acrylate.

According to some of any of the embodiments described herein, the formulation comprises a di-functional alicyclic (meth)acrylate.

According to some of any of the embodiments described herein, the formulation comprises a multi-functional alicyclic acrylate.

According to some of any of the embodiments described herein, the formulation comprises a di-functional alicyclic acrylate, an alicyclic diacrylate.

According to some of any of the embodiments described herein, the formulation comprises a multi-functional (e.g., di-functional) alicyclic (meth)acrylate features, when hardened, high Tg, for example, Tg higher than 100° C.

According to some of any of the embodiments described herein, the formulation comprises a di-functional alicyclic (meth)acrylate featuring, when hardened, high Tg, for example, Tg higher than 100° C.

According to some of any of the embodiments described herein, the formulation comprises a di-functional alicyclic acrylate, or an alicyclic diacrylate, featuring, when hardened, high Tg, for example, Tg higher than 100° C.

According to some of any of the embodiments described herein, the alicyclic diacrylate comprises an alicyclic moiety of at least 6, 7, 8 or more carbon atoms.

According to some of any of the embodiments described herein, the alicyclic diacrylate comprises an alicyclic moiety which comprises 2, 3 or more fused rings.

According to some of any of the embodiments described herein, the multi-functional (meth)acrylate as described herein in any of the respective embodiments features, when hardened, Tg that ranges from 100 to 300, or from 150 to 300, or from 100 to 200, or from 150 to 200,° C., including any intermediate values and subranges therebetween.

According to some of any of the embodiments of this aspect of the present invention, the support material formulation further comprises a photoinitiator, and optionally one or more of a dispersant, an inhibitor, and the like, as described herein in any of the respective embodiments of the modeling material formulation.

According to some embodiments, an amount of the photoinitiator ranges from 0.1 to 1% by weight of the total weight of the formulation.

### Method:

According to an aspect of some embodiments of the present invention there is provided a method of additive manufacturing of a three-dimensional object, as described herein. The method of the present embodiments is usable for manufacturing a denture structure, as defined herein.

The method is generally effected by sequentially forming a plurality of layers in a configured pattern corresponding to the shape of the object, such that formation of each of at least a few of said layers, or of each of said layers, comprises dispensing a building material (uncured) which comprises one or more modeling material formulation(s), and exposing the dispensed modeling material to a curing condition, preferably a curing energy (e.g., irradiation) to thereby form a cured modeling material, as described in further detail hereinafter.

In some exemplary embodiments of the invention an object is manufactured by dispensing a building material (uncured) that comprises two or more different modeling material formulations, for example, as described hereinbelow. In some of these embodiments, each modeling material formulation is dispensed from a different array of nozzles belonging to the same or distinct dispensing heads of the inkjet printing apparatus, as described herein.

In some embodiments, two or more such arrays of nozzles that dispense different modeling material formulations are both located in the same printing head of the AM apparatus (i.e. multi-channels printing head). In some embodiments, arrays of nozzles that dispense different modeling material formulations are located in separate printing heads, for example, a first array of nozzles dispensing a first modeling material formulation is located in a first printing head, and a second array of nozzles dispensing a second modeling material formulation is located in a second printing head.

In some embodiments, an array of nozzles that dispense a modeling material formulation and an array of nozzles that dispense a support material formulation are both located in the same printing head. In some embodiments, an array of nozzles that dispense a modeling material formulation and an array of nozzles that dispense a support material formulation are located in separate printing heads.

The modeling material formulations are optionally and preferably deposited in layers during the same pass of the printing heads. The modeling material formulations and/or combination of formulations within the layer are selected according to the desired properties of the object, and as further described in detail hereinbelow. Such a mode of operation is also referred to herein as “multi-material”

The phrase “digital materials”, as used herein and in the art, describes a combination of two or more materials on a microscopic scale or voxel level such that the printed zones of a specific material are at the level of few voxels, or at a level of a voxel block. Such digital materials may exhibit new properties that are affected by the selection of types of materials and/or the ratio and relative spatial distribution of two or more materials.

In exemplary digital materials, the modeling material of each voxel or voxel block, obtained upon curing, is independent of the modeling material of a neighboring voxel or voxel block, obtained upon curing, such that each voxel or voxel block may result in a different model material and the new properties of the whole part are a result of a spatial combination, on the voxel level, of several different model materials.

The phrase “digital material formulations”, as used herein and in the art, describes a combination of two or more material formulations on a pixel level or voxel level such that pixels or voxels of different material formulations are interlaced with one another over a region. Such digital material formulations may exhibit new properties that are affected by the selection of types of material formulations and/or the ratio and relative spatial distribution of two or more material formulations.

As used herein, a “voxel” of a layer refers to a physical three-dimensional elementary volume within the layer that corresponds to a single pixel of a bitmap describing the layer. The size of a voxel is approximately the size of a region that is formed by a building material, once the building material is dispensed at a location corresponding to the respective pixel, leveled, and solidified.

Herein throughout, whenever the expression “at the voxel level” is used in the context of a different material and/or properties, it is meant to include differences between voxel blocks, as well as differences between voxels or groups of few voxels. In preferred embodiments, the properties of the whole part are a result of a spatial combination, on the voxel block level, of several different model materials.

In some of any of the embodiments of the present invention, once a layer is dispensed as described herein, exposure to a curing condition (e.g., curing energy) as described herein is effected. In some embodiments, the curable materials are photocurable material, preferably UV-curable materials, and the curing condition is such that a radiation source emits UV radiation.

In some of any of the embodiments described herein, the UV irradiation is from a LED source, as described herein.

In some of any of the embodiments described herein, the curing condition comprises electromagnetic irradiation and said electromagnetic irradiation is from a LED source.

In some of any of the embodiments described herein, the curing condition comprises UV irradiation.

In some embodiments, where the building material comprises also support material formulation(s), the method proceeds to removing the hardened support material (e.g., thereby exposing the adjacent hardened modeling material). This can be performed by mechanical and/or chemical means, as would be recognized by any person skilled in the art. A portion of the support material may optionally remain upon removal, for example, within a hardened mixed layer, as described herein.

In some embodiments, removal of hardened support material reveals a hardened mixed layer, comprising a hardened mixture of support material and modeling material formulation. Such a hardened mixture at a surface of an object may optionally have a relatively non-reflective appearance, also referred to herein as “matte”; whereas surfaces lacking such a hardened mixture (e.g., wherein support material formulation was not applied thereon) are described as “glossy” in comparison.

In some of any of the embodiments described herein, the method further comprises exposing the cured modeling material, either before or after (preferably after) removal of a support material, if such has been included in the building material, to a post-treatment condition.

According to some of any of the embodiments described herein, one or more, or all of the dispensed modeling material formulation(s) is/are a modeling material formulation as described herein in any of the respective embodiments and any combination thereof.

According to some of any of the embodiments described herein, the dispensing is of two or more modeling material formulations, each independently being a formulation as described herein in any of the respective embodiments and any combination thereof. In some of these embodiments, the dispensing is of digital materials as described herein.

In exemplary embodiments, the two or more formulation differ from one another by the presence and/or type of a coloring agent and allows manufacturing a single object (e.g., a monolithic denture structure as described herein) featuring a plurality of colors and hues.

According to some of any of the embodiments described herein, the dispensing is further of a support material formulation, for example, a support material formulation as described herein in any of the respective embodiments and any combination thereof.

According to some of any of the embodiments described herein, once the support material is removed, the object is subjected to a post-curing treatment, or post-treatment.

In an exemplary post-treatment procedure, a printed object is subjected to support material removal (e.g., using a water jet), and is then treated sequentially with a solution of a strong base (e.g., sodium hydroxide) (e.g., using a water jet); glycerol (preferably while heating and UV irradiating the object for 2-4 hours; and an alcohol (e.g., isopropyl alcohol). The object can be washed with tap water between being contacted with each of these components. The object is then over-dried or air-dried for at least 2 hours.

According to some of any of the embodiments described herein, the additive manufacturing is three-dimensional inkjet printing.

According to some of any of the embodiments described herein, the denture structure is selected from denture base, an artificial tooth, artificial teeth and a monolithic structure of a denture base and artificial teeth.

According to some of any of the embodiments described herein, the denture structure is a monolithic structure of a denture base and artificial teeth.

### The Object

According to as aspect of some embodiments of the present invention, there is provided a denture structure, as described herein, obtained by additive manufacturing as described herein.

According to some embodiments, the denture structure is a monolithic structure of a denture base and artificial teeth.

According to an aspect of some embodiments of the present invention there is provided a three-dimensional printed object which is a monolithic structure of a denture base and artificial teeth.

According to some of any of the embodiments described herein, the denture structure features mechanical and physical properties in accordance with the requirements of ISO 20795-1 and ISO 10477 and biocompatibility properties in accordance with the requirements of ISO 10993-1.

### System:

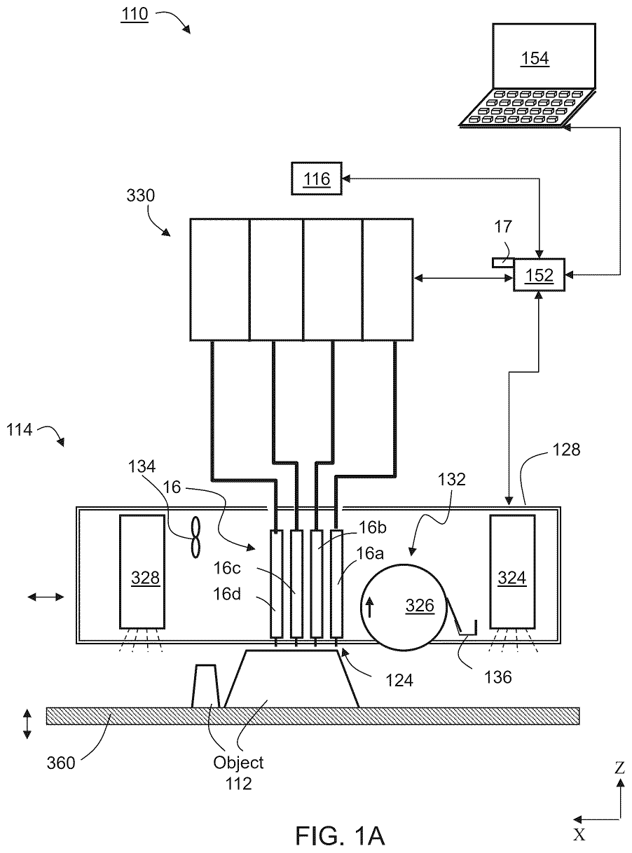

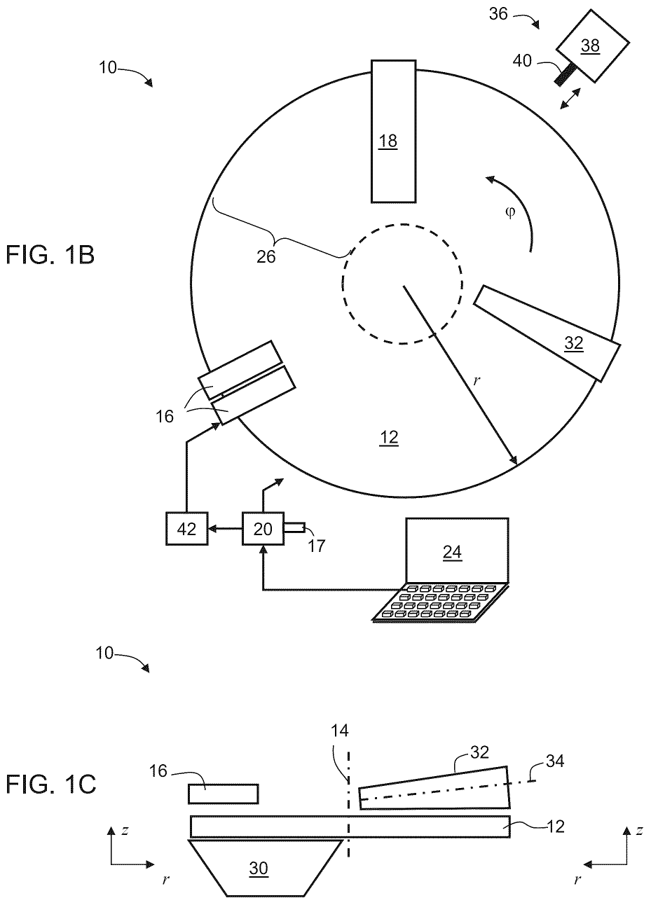

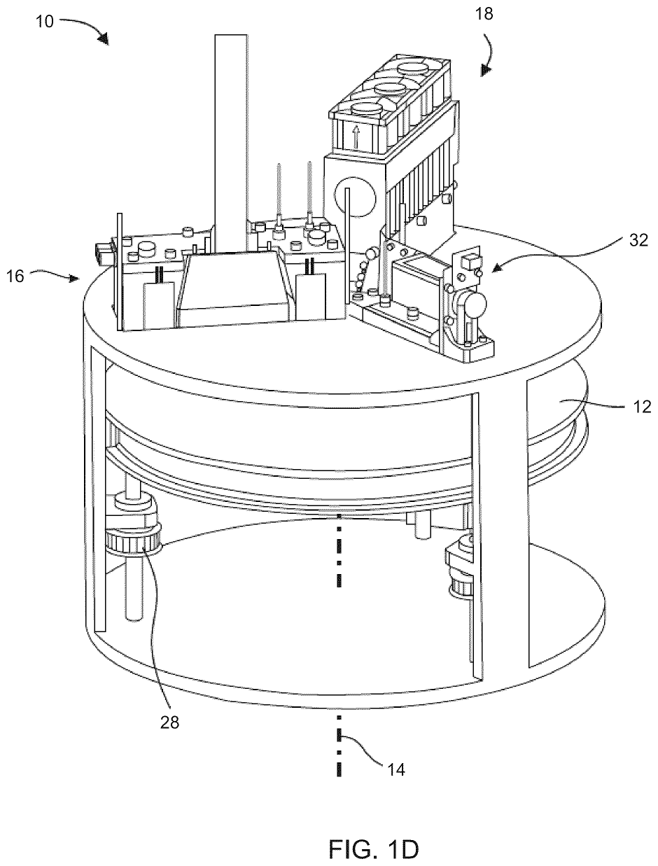

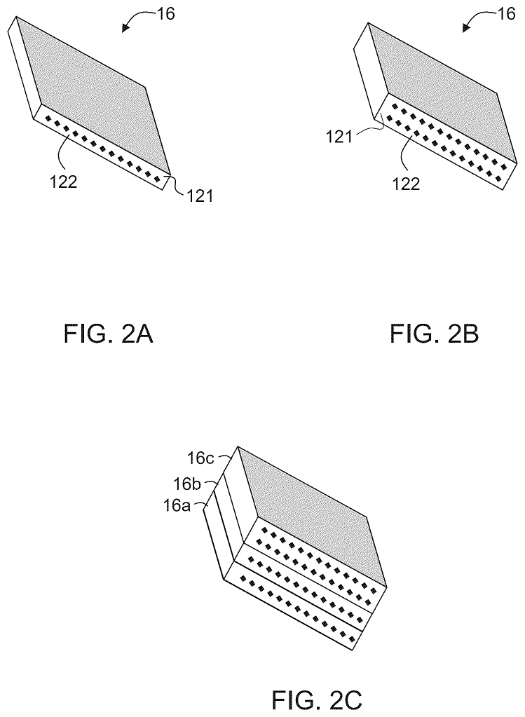

A representative and non-limiting example of a system 110 suitable for AM of an object 112 according to some embodiments of the present invention is illustrated in FIG. 1A. System 110 comprises an additive manufacturing apparatus 114 having a dispensing unit 16 which comprises a plurality of printing heads. Each head preferably comprises one or more arrays of nozzles 122, typically mounted on an orifice plate 121, as illustrated in FIGS. 2A-C described below, through which a liquid building material formulation 124 is dispensed.

Preferably, but not obligatorily, apparatus 114 is a three-dimensional printing apparatus, in which case the printing heads are printing heads, and the building material formulation is dispensed via inkjet technology. This need not necessarily be the case, since, for some applications, it may not be necessary for the additive manufacturing apparatus to employ three-dimensional printing techniques. Representative examples of additive manufacturing apparatus contemplated according to various exemplary embodiments of the present invention include, without limitation, fused deposition modeling apparatus and fused material formulation deposition apparatus.

Each printing head is optionally and preferably fed via one or more building material formulation reservoirs which may optionally include a temperature control unit (e.g., a temperature sensor and/or a heating device), and a material formulation level sensor. To dispense the building material formulation, a voltage signal is applied to the printing heads to selectively deposit droplets of material formulation via the printing head nozzles, for example, as in piezoelectric inkjet printing technology. Another example includes thermal inkjet printing heads. In these types of heads, there are heater elements in thermal contact with the building material formulation, for heating the building material formulation to form gas bubbles therein, upon activation of the heater elements by a voltage signal. The gas bubbles generate pressures in the building material formulation, causing droplets of building material formulation to be ejected through the nozzles. Piezoelectric and thermal printing heads are known to those skilled in the art of solid freeform fabrication. For any types of inkjet printing heads, the dispensing rate of the head depends on the number of nozzles, the type of nozzles and the applied voltage signal rate (frequency).

Optionally, the overall number of dispensing nozzles or nozzle arrays is selected such that half of the dispensing nozzles are designated to dispense support material formulation and half of the dispensing nozzles are designated to dispense modeling material formulation, i.e. the number of nozzles jetting modeling material formulations is the same as the number of nozzles jetting support material formulation. In the representative example of FIG. 1A, four printing heads 16a, 16b, 16c and 16d are illustrated. Each of heads 16a, 16b, 16c and 16d has a nozzle array. In this Example, heads 16a and 16b can be designated for modeling material formulation/s and heads 16c and 16d can be designated for support material formulation. Thus, head 16a can dispense one modeling material formulation, head 16b can dispense another modeling material formulation and heads 16c and 16d can both dispense support material formulation. In an alternative embodiment, heads 16c and 16d, for example, may be combined in a single head having two nozzle arrays for depositing support material formulation. In a further alternative embodiment any one or more of the printing heads may have more than one nozzle arrays for depositing more than one material formulation, e.g. two nozzle arrays for depositing two different modeling material formulations or a modeling material formulation and a support material formulation, each formulation via a different array or number of nozzles.

Yet it is to be understood that it is not intended to limit the scope of the present invention and that the number of modeling material formulation printing heads (modeling heads) and the number of support material formulation printing heads (support heads) may differ. Generally, the number of arrays of nozzles that dispense modeling material formulation, the number of arrays of nozzles that dispense support material formulation, and the number of nozzles in each respective array are selected such as to provide a predetermined ratio, a, between the maximal dispensing rate of the support material formulation and the maximal dispensing rate of modeling material formulation. The value of the predetermined ratio, a, is preferably selected to ensure that in each formed layer, the height of modeling material formulation equals the height of support material formulation. Typical values for a are from about 0.6 to about 1.5.

As used herein throughout the term “about” refers to +10%.

For example, for a ═1, the overall dispensing rate of support material formulation is generally the same as the overall dispensing rate of the modeling material formulation when all the arrays of nozzles operate.

Apparatus 114 can comprise, for example, M modeling heads each having m arrays of p nozzles, and S support heads each having s arrays of q nozzles such that M×m×p=S×s×q. Each of the M×m modeling arrays and S×s support arrays can be manufactured as a separate physical unit, which can be assembled and disassembled from the group of arrays. In this embodiment, each such array optionally and preferably comprises a temperature control unit and a material formulation level sensor of its own, and receives an individually controlled voltage for its operation.

Apparatus 114 can further comprise a solidifying device 324 which can include any device configured to emit light, heat or the like that may cause the deposited material formulation to harden. For example, solidifying device 324 can comprise one or more radiation sources, which can be, for example, an ultraviolet or visible or infrared lamp, or other sources of electromagnetic radiation, or electron beam source, depending on the modeling material formulation being used. In some embodiments of the present invention, solidifying device 324 serves for curing or solidifying the modeling material formulation.

In addition to solidifying device 324, apparatus 114 optionally and preferably comprises an additional radiation source 328 for solvent evaporation. Radiation source 328 optionally and preferably generates infrared radiation. In various exemplary embodiments of the invention solidifying device 324 comprises a radiation source generating ultraviolet radiation, and radiation source 328 generates infrared radiation.

In some embodiments of the present invention apparatus 114 comprises cooling system 134 such as one or more fans or the like

The printing head(s) and radiation source are preferably mounted in a frame or block 128 which is preferably operative to reciprocally move over a tray 360, which serves as the working surface. In some embodiments of the present invention the radiation sources are mounted in the block such that they follow in the wake of the printing heads to at least partially cure or solidify the material formulations just dispensed by the printing heads. Tray 360 is positioned horizontally. According to the common conventions an X-Y-Z Cartesian coordinate system is selected such that the X-Y plane is parallel to tray 360. Tray 360 is preferably configured to move vertically (along the Z direction), typically downward. In various exemplary embodiments of the invention, apparatus 114 further comprises one or more leveling devices 132, e.g. a roller 326. Leveling device 326 serves to straighten, level and/or establish a thickness of the newly formed layer prior to the formation of the successive layer thereon. Leveling device 326 preferably comprises a waste collection device 136 for collecting the excess material formulation generated during leveling. Waste collection device 136 may comprise any mechanism that delivers the material formulation to a waste tank or waste cartridge.

In use, the printing heads of unit 16 move in a scanning direction, which is referred to herein as the X direction, and selectively dispense building material formulation in a predetermined configuration in the course of their passage over tray 360. The building material formulation typically comprises one or more types of support material formulation and one or more types of modeling material formulation. The passage of the printing heads of unit 16 is followed by the curing of the modeling material formulation(s) by radiation source 126. In the reverse passage of the heads, back to their starting point for the layer just deposited, an additional dispensing of building material formulation may be carried out, according to predetermined configuration. In the forward and/or reverse passages of the printing heads, the layer thus formed may be straightened by leveling device 326, which preferably follows the path of the printing heads in their forward and/or reverse movement. Once the printing heads return to their starting point along the X direction, they may move to another position along an indexing direction, referred to herein as the Y direction, and continue to build the same layer by reciprocal movement along the X direction. Alternately, the printing heads may move in the Y direction between forward and reverse movements or after more than one forward-reverse movement. The series of scans performed by the printing heads to complete a single layer is referred to herein as a single scan cycle.

Once the layer is completed, tray 360 is lowered in the Z direction to a predetermined Z level, according to the desired thickness of the layer subsequently to be printed. The procedure is repeated to form three-dimensional object 112 in a layer-wise manner.

In another embodiment, tray 360 may be displaced in the Z direction between forward and reverse passages of the printing head of unit 16, within the layer. Such Z displacement is carried out in order to cause contact of the leveling device with the surface in one direction and prevent contact in the other direction.

System 110 optionally and preferably comprises a building material formulation supply system 330 which comprises the building material formulation containers or cartridges and supplies a plurality of building material formulations to fabrication apparatus 114.

A control unit 152 controls fabrication apparatus 114 and optionally and preferably also supply system 330. Control unit 152 typically includes an electronic circuit configured to perform the controlling operations. Control unit 152 preferably communicates with a data processor 154 which transmits digital data pertaining to fabrication instructions based on computer object data, e.g., a CAD configuration represented on a computer readable medium in a form of a Standard Tessellation Language (STL) format or the like. Typically, control unit 152 controls the voltage applied to each printing head or each nozzle array and the temperature of the building material formulation in the respective printing head or respective nozzle array.

Once the manufacturing data is loaded to control unit 152 it can operate without user intervention. In some embodiments, control unit 152 receives additional input from the operator, e.g., using data processor 154 or using a user interface 116 communicating with unit 152. User interface 116 can be of any type known in the art, such as, but not limited to, a keyboard, a touch screen and the like. For example, control unit 152 can receive, as additional input, one or more building material formulation types and/or attributes, such as, but not limited to, color, characteristic distortion and/or transition temperature, viscosity, electrical property, magnetic property. Other attributes and groups of attributes are also contemplated.

Another representative and non-limiting example of a system 10 suitable for AM of an object according to some embodiments of the present invention is illustrated in FIGS. 1B-D. FIGS. 1B-D illustrate a top view (FIG. 1B), a side view (FIG. 1C) and an isometric view (FIG. 1D) of system 10.

In the present embodiments, system 10 comprises a tray 12 and a plurality of inkjet printing heads 16, each having one or more arrays of nozzles with respective one or more pluralities of separated nozzles. The material used for the three-dimensional printing is supplied to heads 16 by a building material supply system 42. Tray 12 can have a shape of a disk or it can be annular. Non-round shapes are also contemplated, provided they can be rotated about a vertical axis.

Tray 12 and heads 16 are optionally and preferably mounted such as to allow a relative rotary motion between tray 12 and heads 16. This can be achieved by (i) configuring tray 12 to rotate about a vertical axis 14 relative to heads 16, (ii) configuring heads 16 to rotate about vertical axis 14 relative to tray 12, or (iii) configuring both tray 12 and heads 16 to rotate about vertical axis 14 but at different rotation velocities (e.g., rotation at opposite direction). While some embodiments of system 10 are described below with a particular emphasis to configuration (i) wherein the tray is a rotary tray that is configured to rotate about vertical axis 14 relative to heads 16, it is to be understood that the present application contemplates also configurations (ii) and (iii) for system 10. Any one of the embodiments of system 10 described herein can be adjusted to be applicable to any of configurations (ii) and (iii), and one of ordinary skills in the art, provided with the details described herein, would know how to make such adjustment.

In the following description, a direction parallel to tray 12 and pointing outwardly from axis 14 is referred to as the radial direction r, a direction parallel to tray 12 and perpendicular to the radial direction r is referred to herein as the azimuthal direction o, and a direction perpendicular to tray 12 is referred to herein is the vertical direction z.

The radial direction r in system 10 enacts the indexing direction y in system 110, and the azimuthal direction o enacts the scanning direction x in system 110. Therefore, the radial direction is interchangeably referred to herein as the indexing direction, and the azimuthal direction is interchangeably referred to herein as the scanning direction.

The term “radial position,” as used herein, refers to a position on or above tray 12 at a specific distance from axis 14. When the term is used in connection to a printing head, the term refers to a position of the head which is at specific distance from axis 14. When the term is used in connection to a point on tray 12, the term corresponds to any point that belongs to a locus of points that is a circle whose radius is the specific distance from axis 14 and whose center is at axis 14.

The term “azimuthal position,” as used herein, refers to a position on or above tray 12 at a specific azimuthal angle relative to a predetermined reference point. Thus, radial position refers to any point that belongs to a locus of points that is a straight line forming the specific azimuthal angle relative to the reference point.

The term “vertical position,” as used herein, refers to a position over a plane that intersect the vertical axis 14 at a specific point.

Tray 12 serves as a building platform for three-dimensional printing. The working area on which one or objects are printed is typically, but not necessarily, smaller than the total area of tray 12. In some embodiments of the present invention the working area is annular. The working area is shown at 26. In some embodiments of the present invention tray 12 rotates continuously in the same direction throughout the formation of object, and in some embodiments of the present invention tray reverses the direction of rotation at least once (e.g., in an oscillatory manner) during the formation of the object. Tray 12 is optionally and preferably removable. Removing tray 12 can be for maintenance of system 10, or, if desired, for replacing the tray before printing a new object.

In some embodiments of the present invention system 10 is provided with one or more different replacement trays (e.g., a kit of replacement trays), wherein two or more trays are designated for different types of objects (e.g., different weights) different operation modes (e.g., different rotation speeds), etc. The replacement of tray 12 can be manual or automatic, as desired. When automatic replacement is employed, system 10 comprises a tray replacement device 36 configured for removing tray 12 from its position below heads 16 and replacing it by a replacement tray (not shown). In the representative illustration of FIG. 1B tray replacement device 36 is illustrated as a drive 38 with a movable arm 40 configured to pull tray 12, but other types of tray replacement devices are also contemplated.

Exemplified embodiments for the printing head 16 are illustrated in FIGS. 2A-2C. These embodiments can be employed for any of the AM systems described above, including, without limitation, system 110 and system 10.

FIGS. 2A-B illustrate a printing head 16 with one (FIG. 2A) and two (FIG. 2B) nozzle arrays 22. The nozzles in the array are preferably aligned linearly, along a straight line. In embodiments in which a particular printing head has two or more linear nozzle arrays, the nozzle arrays are optionally and preferably can be parallel to each other. When a printing head has two or more arrays of nozzles (e.g., FIG. 2B) all arrays of the head can be fed with the same building material formulation, or at least two arrays of the same head can be fed with different building material formulations.

When a system similar to system 110 is employed, all printing heads 16 are optionally and preferably oriented along the indexing direction with their positions along the scanning direction being offset to one another.

When a system similar to system 10 is employed, all printing heads 16 are optionally and preferably oriented radially (parallel to the radial direction) with their azimuthal positions being offset to one another. Thus, in these embodiments, the nozzle arrays of different printing heads are not parallel to each other but are rather at an angle to each other, which angle being approximately equal to the azimuthal offset between the respective heads. For example, one head can be oriented radially and positioned at azimuthal position φ₁, and another head can be oriented radially and positioned at azimuthal position φ₂. In this example, the azimuthal offset between the two heads is φ₁-φ₂, and the angle between the linear nozzle arrays of the two heads is also φ₁-φ₂.

In some embodiments, two or more printing heads can be assembled to a block of printing heads, in which case the printing heads of the block are typically parallel to each other. A block including several inkjet printing heads 16a, 16b, 16c is illustrated in FIG. 2C.

In some embodiments, system 10 comprises a stabilizing structure 30 positioned below heads 16 such that tray 12 is between stabilizing structure 30 and heads 16. Stabilizing structure 30 may serve for preventing or reducing vibrations of tray 12 that may occur while inkjet printing heads 16 operate. In configurations in which printing heads 16 rotate about axis 14, stabilizing structure 30 preferably also rotates such that stabilizing structure 30 is always directly below heads 16 (with tray 12 between heads 16 and tray 12).

Tray 12 and/or printing heads 16 is optionally and preferably configured to move along the vertical direction z, parallel to vertical axis 14 so as to vary the vertical distance between tray 12 and printing heads 16. In configurations in which the vertical distance is varied by moving tray 12 along the vertical direction, stabilizing structure 30 preferably also moves vertically together with tray 12. In configurations in which the vertical distance is varied by heads 16 along the vertical direction, while maintaining the vertical position of tray 12 fixed, stabilizing structure 30 is also maintained at a fixed vertical position.

The vertical motion can be established by a vertical drive 28. Once a layer is completed, the vertical distance between tray 12 and heads 16 can be increased (e.g., tray 12 is lowered relative to heads 16) by a predetermined vertical step, according to the desired thickness of the layer subsequently to be printed. The procedure is repeated to form a three-dimensional object in a layer-wise manner.

The operation of inkjet printing heads 16 and optionally and preferably also of one or more other components of system 10, e.g., the motion of tray 12, are controlled by a controller 20. The controller can have an electronic circuit and a non-volatile memory medium readable by the circuit, wherein the memory medium stores program instructions which, when read by the circuit, cause the circuit to perform control operations as further detailed below.

Controller 20 can also communicate with a host computer 24 which transmits digital data pertaining to fabrication instructions based on computer object data, e.g., in a form of a Standard Tessellation Language (STL) or a StereoLithography Contour (SLC) format, Virtual Reality Modeling Language (VRML), Additive Manufacturing File (AMF) format, Drawing Exchange Format (DXF), Polygon File Format (PLY) or any other format suitable for Computer-Aided Design (CAD). The object data formats are typically structured according to a Cartesian system of coordinates. In these cases, computer 24 preferably executes a procedure for transforming the coordinates of each slice in the computer object data from a Cartesian system of coordinates into a polar system of coordinates. Computer 24 optionally and preferably transmits the fabrication instructions in terms of the transformed system of coordinates. Alternatively, computer 24 can transmit the fabrication instructions in terms of the original system of coordinates as provided by the computer object data, in which case the transformation of coordinates is executed by the circuit of controller 20.

The transformation of coordinates allows three-dimensional printing over a rotating tray. In non-rotary systems with a stationary tray with the printing heads typically reciprocally move above the stationary tray along straight lines. In such systems, the printing resolution is the same at any point over the tray, provided the dispensing rates of the heads are uniform. In system 10, unlike non-rotary systems, not all the nozzles of the head points cover the same distance over tray 12 during at the same time. The transformation of coordinates is optionally and preferably executed so as to ensure equal amounts of excess material formulation at different radial positions.

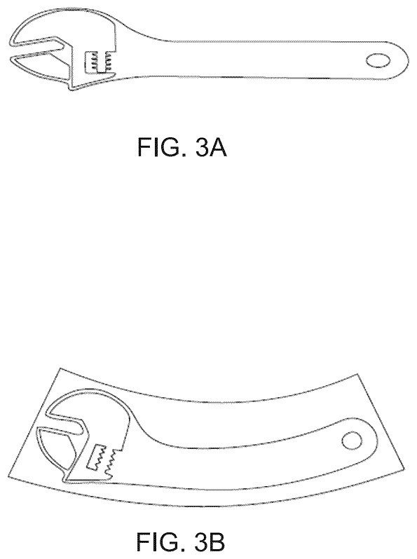

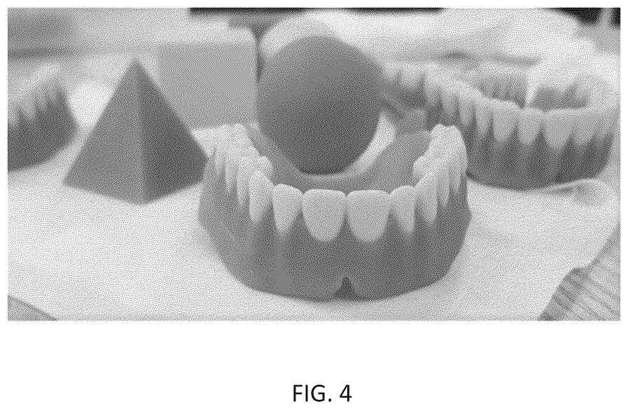

Representative examples of coordinate transformations according to some embodiments of the present invention are provided in FIGS. 3A-B, showing three slices of an object (each slice corresponds to fabrication instructions of a different layer of the objects), where FIG. 3A illustrates a slice in a Cartesian system of coordinates and FIG. 3B illustrates the same slice following an application of a transformation of coordinates procedure to the respective slice.

Typically, controller 20 controls the voltage applied to the respective component of the system 10 based on the fabrication instructions and based on the stored program instructions as described below.

Generally, controller 20 controls printing heads 16 to dispense, during the rotation of tray 12, droplets of building material formulation in layers, such as to print a three-dimensional object on tray 12.

System 10 optionally and preferably comprises one or more radiation sources 18, which can be, for example, an ultraviolet or visible or infrared lamp, or other sources of electromagnetic radiation, or electron beam source, depending on the modeling material formulation being used. Radiation source can include any type of radiation emitting device, including, without limitation, light emitting diode (LED), digital light processing (DLP) system, resistive lamp and the like. Radiation source 18 serves for curing or solidifying the modeling material formulation. In various exemplary embodiments of the invention the operation of radiation source 18 is controlled by controller 20 which may activate and deactivate radiation source 18 and may optionally also control the amount of radiation generated by radiation source 18.

In some embodiments of the invention, system 10 further comprises one or more leveling devices 32 which can be manufactured as a roller or a blade. Leveling device 32 serves to straighten the newly formed layer prior to the formation of the successive layer thereon. In some embodiments, leveling device 32 has the shape of a conical roller positioned such that its symmetry axis 34 is tilted relative to the surface of tray 12 and its surface is parallel to the surface of the tray. This embodiment is illustrated in the side view of system 10 (FIG. 1C).

The conical roller can have the shape of a cone or a conical frustum.

The opening angle of the conical roller is preferably selected such that there is a constant ratio between the radius of the cone at any location along its axis 34 and the distance between that location and axis 14. This embodiment allows roller 32 to efficiently level the layers, since while the roller rotates, any point p on the surface of the roller has a linear velocity which is proportional (e.g., the same) to the linear velocity of the tray at a point vertically beneath point p. In some embodiments, the roller has a shape of a conical frustum having a height h, a radius R₁ at its closest distance from axis 14, and a radius R₂ at its farthest distance from axis 14, wherein the parameters h, R₁ and R₂ satisfy the relation R₁/R₂=(R−h)/h and wherein R is the farthest distance of the roller from axis 14 (for example, R can be the radius of tray 12).

The operation of leveling device 32 is optionally and preferably controlled by controller 20 which may activate and deactivate leveling device 32 and may optionally also control its position along a vertical direction (parallel to axis 14) and/or a radial direction (parallel to tray 12 and pointing toward or away from axis 14.

In some embodiments of the present invention printing heads 16 are configured to reciprocally move relative to tray along the radial direction r. These embodiments are useful when the lengths of the nozzle arrays 22 of heads 16 are shorter than the width along the radial direction of the working area 26 on tray 12. The motion of heads 16 along the radial direction is optionally and preferably controlled by controller 20.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Herein the terms “method” and “process” are used interchangeably and refer to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein throughout, whenever the phrase “weight percent”, or “% by weight” or “% wt.”, is indicated in the context of embodiments of a formulation (e.g., a modeling formulation), it is meant weight percent of the total weight of the respective uncured formulation.

Herein throughout, an acrylic material is used to collectively describe material featuring one or more acrylate, methacrylate, acrylamide and/or methacrylamide group(s).

Similarly, an acrylic group is used to collectively describe curable groups which are acrylate, methacrylate, acrylamide and/or methacrylamide group(s), preferably acrylate or methacrylate groups (referred to herein also as (meth)acrylate groups).

Herein throughout, the term “(meth)acrylic” encompasses acrylic and methacrylic materials.

Herein throughout, the phrase “linking moiety” or “linking group” describes a group that connects two or more moieties or groups in a compound. A linking moiety is typically derived from a bi- or tri-functional compound, and can be regarded as a bi- or tri-radical moiety, which is connected to two or three other moieties, via two or three atoms thereof, respectively.

Exemplary linking moieties include a hydrocarbon moiety or chain, optionally interrupted by one or more heteroatoms, as defined herein, and/or any of the chemical groups listed below, when defined as linking groups.

When a chemical group is referred to herein as “end group” it is to be interpreted as a substituent, which is connected to another group via one atom thereof.

Herein throughout, the term “hydrocarbon” collectively describes a chemical group composed mainly of carbon and hydrogen atoms. A hydrocarbon can be comprised of alkyl, alkene, alkyne, aryl, and/or cycloalkyl, each can be substituted or unsubstituted, and can be interrupted by one or more heteroatoms. The number of carbon atoms can range from 2 to 30, and is preferably lower, e.g., from 1 to 10, or from 1 to 6, or from 1 to 4. A hydrocarbon can be a linking group or an end group.

Bisphenol A is an example of a hydrocarbon comprised of 2 aryl groups and one alkyl group. Dimethylenecyclohexane is an example of a hydrocarbon comprised of 2 alkyl groups and one cycloalkyl group.

As used herein, the term “amine” describes both a —NR′R″ group and a —NR′-group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

The term “amine” is used herein to describe a —NR′R″ group in cases where the amine is an end group, as defined hereinunder, and is used herein to describe a —NR′-group in cases where the amine is a linking group or is or part of a linking moiety.

The term “alkyl” describes a saturated aliphatic hydrocarbon including straight chain and branched chain groups. Preferably, the alkyl group has 1 to 30, or 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking group, as this phrase is defined hereinabove, which connects two or more moieties via at least two carbons in its chain. When the alkyl is a linking group, it is also referred to herein as “alkylene” or “alkylene chain”.

Alkene and Alkyne, as used herein, are an alkyl, as defined herein, which contains one or more double bond or triple bond, respectively.

The term “cycloalkyl” describes an all-carbon monocyclic ring or fused rings (i.e., rings which share an adjacent pair of carbon atoms) group where one or more of the rings does not have a completely conjugated pi-electron system. Examples include, without limitation, cyclohexane, adamantine, norbornyl, isobornyl, and the like. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking group, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino, oxalidine, and the like.

The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking group, as this phrase is defined hereinabove, connecting two or more moieties at two or more positions thereof.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking group, as this term is defined hereinabove, connecting two or more moieties at two or more positions thereof.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, oxazole, indole, purine and the like.

The term “halide” and “halo” describes fluorine, chlorine, bromine or iodine. The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking group, as these phrases are defined hereinabove, where R′ is as defined hereinabove.

The term “thiosulfate” describes a —O—S(═S)(═O)—OR′ end group or a —O—S(═S)(═O)—O— linking group, as these phrases are defined hereinabove, where R′ is as defined hereinabove.

The term “sulfite” describes an —O—S(═O)—O—R′ end group or a —O—S(═O)—O— group linking group, as these phrases are defined hereinabove, where R′ is as defined hereinabove.

The term “thiosulfite” describes a —O—S(═S)—O—R′ end group or an —O—S(═S)—O— group linking group, as these phrases are defined hereinabove, where R′ is as defined hereinabove.

The term “sulfinate” describes a —S(═O)—OR′ end group or an —S(═O)—O— group linking group, as these phrases are defined hereinabove, where R′ is as defined hereinabove.

The term “sulfoxide” or “sulfinyl” describes a —S(═O)R′ end group or an —S(═O)— linking group, as these phrases are defined hereinabove, where R′ is as defined hereinabove.

The term “sulfonate” describes a —S(═O)₂—R′ end group or an —S(═O)₂— linking group, as these phrases are defined hereinabove, where R′ is as defined herein.

The term “S-sulfonamide” describes a —S(═O)₂—NR′R″ end group or a —S(═O)₂—NR′— linking group, as these phrases are defined hereinabove, with R′ and R″ as defined herein.

The term “N-sulfonamide” describes an R'S(═O)₂—NR″-end group or a —S(═O)₂—NR′— linking group, as these phrases are defined hereinabove, where R′ and R″ are as defined herein.

The term “disulfide” refers to a —S—SR′ end group or a —S—S— linking group, as these phrases are defined hereinabove, where R′ is as defined herein.

The term “phosphonate” describes a —P(═O)(OR′)(OR″) end group or a —P(═O)(OR′)(O)— linking group, as these phrases are defined hereinabove, with R′ and R″ as defined herein.

The term “thiophosphonate” describes a —P(═S)(OR′)(OR″) end group or a —P(═S)(OR′)(O)— linking group, as these phrases are defined hereinabove, with R′ and R″ as defined herein.

The term “phosphinyl” describes a —PR′R″ end group or a —PR′— linking group, as these phrases are defined hereinabove, with R′ and R″ as defined hereinabove.

The term “phosphine oxide” describes a —P(═O)(R′)(R″) end group or a —P(═O)(R′)— linking group, as these phrases are defined hereinabove, with R′ and R″ as defined herein.

The term “phosphine sulfide” describes a —P(═S)(R′)(R″) end group or a —P(═S)(R′)— linking group, as these phrases are defined hereinabove, with R′ and R″ as defined herein.

The term “phosphite” describes an —O—PR′(═O)(OR″) end group or an —O—PH(═O)(O)— linking group, as these phrases are defined hereinabove, with R′ and R″ as defined herein.

The term “carbonyl” or “carbonate” as used herein, describes a —C(═O)—R′ end group or a —C(═O)— linking group, as these phrases are defined hereinabove, with R′ as defined herein.

The term “thiocarbonyl” as used herein, describes a —C(═S)—R′ end group or a —C(═S)— linking group, as these phrases are defined hereinabove, with R′ as defined herein.

The term “oxo” as used herein, describes a (═O) group, wherein an oxygen atom is linked by a double bond to the atom (e.g., carbon atom) at the indicated position.

The term “thiooxo” as used herein, describes a (═S) group, wherein a sulfur atom is linked by a double bond to the atom (e.g., carbon atom) at the indicated position.

The term “oxime” describes a ═N—OH end group or a ═N—O— linking group, as these phrases are defined hereinabove.

The term “hydroxyl” describes a —OH group.

The term “alkoxy” describes both an —O-alkyl and an —O-cycloalkyl group, as defined herein. The term alkoxide describes-R′O-group, with R′ as defined herein.

The term “aryloxy” describes both an —O-aryl and an —O-heteroaryl group, as defined herein.

The term “thiohydroxy” or “thiol” describes a —SH group. The term “thiolate” describes a —S⁻ group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The “hydroxyalkyl” is also referred to herein as “alcohol”, and describes an alkyl, as defined herein, substituted by a hydroxy group.

The term “cyano” describes a —C═N group.

The term “isocyanate” describes an —N═C═O group.

The term “isothiocyanate” describes an —N═C═S group.

The term “nitro” describes an —NO₂ group.

The term “acyl halide” describes a —(C═O)R″ group wherein R″″ is halide, as defined hereinabove.

The term “azo” or “diazo” describes an —N═NR′ end group or an —N═N— linking group, as these phrases are defined hereinabove, with R′ as defined hereinabove.

The term “peroxo” describes an —O—OR′ end group or an —O—O— linking group, as these phrases are defined hereinabove, with R′ as defined hereinabove.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone. Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and O-thiocarboxylate.

The term “C-thiocarboxylate” describes a —C(═S)—OR′ end group or a —C(═S)—O— linking group, as these phrases are defined hereinabove, where R′ is as defined herein.

The term “O-thiocarboxylate” describes a —OC(═S) R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone. Alternatively, R′ and O are linked together to form a ring in O-thiocarboxylate. Cyclic thiocarboxylates can function as a linking group, for example, when an atom in the formed ring is linked to another group.

The term “carbamate” as used herein encompasses N-carbamate and O-carbamate.

The term “N-carbamate” describes an R″OC(═O)—NR′-end group or a —OC(═O)—NR′— linking group, as these phrases are defined hereinabove, with R′ and R″ as defined herein.

The term “O-carbamate” describes an —OC(═O)—NR′R″ end group or an —OC(═O)-NR′— linking group, as these phrases are defined hereinabove, with R′ and R″ as defined herein.

A carbamate can be linear or cyclic. When cyclic, R′ and the carbon atom are linked together to form a ring, in O-carbamate. Alternatively, R′ and O are linked together to form a ring in N-carbamate. Cyclic carbamates can function as a linking group, for example, when an atom in the formed ring is linked to another group.

The term “carbamate” as used herein encompasses N-carbamate and O-carbamate.

The term “thiocarbamate” as used herein encompasses N-thiocarbamate and O-thiocarbamate. describes a —OC(═S)—NR′R″ end group or

The term “O-thiocarbamate” a —OC(═S)—NR′— linking group, as these phrases are defined hereinabove, with R′ and R″ as defined herein.

The term “N-thiocarbamate” describes an R″OC(═S)NR′-end group or a —OC(═S)NR′-linking group, as these phrases are defined hereinabove, with R′ and R″ as defined herein.

Thiocarbamates can be linear or cyclic, as described herein for carbamates.

The term “dithiocarbamate” as used herein encompasses S-dithiocarbamate and N-dithiocarbamate.

The term “S-dithiocarbamate” describes a —SC(═S)—NR′R″ end group or a-SC(═S)NR′— linking group, as these phrases are defined hereinabove, with R′ and R″ as defined herein.

The term “N-dithiocarbamate” describes an R″SC(═S)NR′-end group or a —SC(═S)NR′-linking group, as these phrases are defined hereinabove, with R′ and R″ as defined herein.

The term “urea”, which is also referred to herein as “ureido”, describes a —NR′C(═O)—NR″R′″ end group or a —NR′C(—O)—NR″— linking group, as these phrases are defined hereinabove, where R′ and R″ are as defined herein and R″ is as defined herein for R′ and R″. The term “thiourea”, which is also referred to herein as “thioureido”, describes a —NR′—C(═S)—NR″R′″ end group or a —NR′—C(═S)—NR″— linking group, with R′, R″ and R′″ as defined herein.

The term “amide” as used herein encompasses C-amide and N-amide.

The term “C-amide” describes a —C(═O)—NR′R″ end group or a —C(═O)—NR′— linking group, as these phrases are defined hereinabove, where R′ and R″ are as defined herein.

The term “N-amide” describes a R′C(═O)—NR″-end group or a R′C(═O)—N— linking group, as these phrases are defined hereinabove, where R′ and R″ are as defined herein.

An amide can be linear or cyclic. When cyclic, R′ and the carbon atom are linked together to form a ring, in C-amide, and this group is also referred to as lactam. Cyclic amides can function as a linking group, for example, when an atom in the formed ring is linked to another group.

The term “guanyl” describes a R′R″NC(═N)-end group or a —R′NC(═N)— linking group, as these phrases are defined hereinabove, where R′ and R″ are as defined herein.

The term “guanidine” describes a —R′NC(═N)—NR″R′″ end group or a —R′NC(═N)—NR″— linking group, as these phrases are defined hereinabove, where R′, R″ and R″ are as defined herein.

The term “hydrazine” describes a —NR′—NR″R′″ end group or a —NR′—NR″— linking group, as these phrases are defined hereinabove, with R′, R″, and R″ as defined herein.

As used herein, the term “hydrazide” describes a —C(═O)—NR′—NR″R′″ end group or a —C(═O)—NR′—NR″— linking group, as these phrases are defined hereinabove, where R′, R″ and R′″ are as defined herein.

As used herein, the term “thiohydrazide” describes a —C(═S)—NR′—NR″R′″ end group or a —C(═S)—NR′—NR″— linking group, as these phrases are defined hereinabove, where R′, R″ and R′″ are as defined herein.

The term “cyanurate” describes an

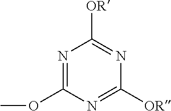

end group or

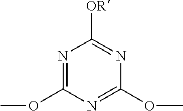

linking group, with R′ and R″ as defined herein.

The term “isocyanurate” describes an

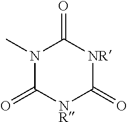

end group or a

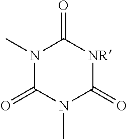

The term “thiocyanurate” describes an

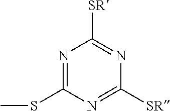

end group or

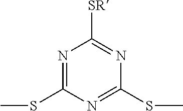

linking group, with R′ and R″ as defined herein.

As used herein, the term “alkylene glycol” describes a —O—[(CR′R″)_{z}—O]_{y}—R′″ end group or a —O—[(CR′R″)_{z}—O]_{y}— linking group, with R′, R″ and R′″ being as defined herein, and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is 2-4, the alkylene glycol is referred to herein as oligo (alkylene glycol).

Herein, an “ethoxylated” material describes an acrylic or methacrylic compound which comprises one or more alkylene glycol groups, or, preferably, one or more alkylene glycol chains, as defined herein. Ethoxylated (meth)acrylate materials can be mono-functional, or, preferably, multi-functional, namely, di-functional, tri-functional, tetrafunctional, etc.

In multi-functional materials, typically, each of the (meth)acrylate groups are linked to an alkylene glycol group or chain, and the alkylene glycol groups or chains are linked to one another through a branching unit, such as, for example, a branched alkyl, cycloalkyl, aryl (e.g., Bisphenol A), etc.

In some embodiments, the ethoxylated material comprises at least one, or at least two ethoxylated group(s), that is, at least one or at least two alkylene glycol moieties or groups. Some or all of the alkylene glycol groups can be linked to one another to form an alkylene glycol chain. For example, an ethoxylated material that comprises 30 ethoxylated groups can comprise a chain of 30 alkylene glycol groups linked to one another, two chains, each, for example, of 15 alkylene glycol moieties linked to one another, the two chains linked to one another via a branching moiety, or three chains, each, for example, of 10 alkylene glycol groups linked to one another, the three chains linked to one another via a branching moiety. Shorter and longer chains are also contemplated.

The ethoxylated material can comprise one, two or more alkylene glycol chains, of any length.

The term “branching unit” as used herein describes a multi-radical, preferably aliphatic or alicyclic group. By “multi-radical” it is meant that the unit has two or more attachment points such that it links between two or more atoms and/or groups or moieties.

In some embodiments, the branching unit is derived from a chemical moiety that has two, three or more functional groups. In some embodiments, the branching unit is a branched alkyl or a cycloalkyl (alicyclic) or an aryl (e.g., phenyl) as defined herein.

As used herein, the phrase “impact resistance”, which is also referred to interchangeably, herein and in the art, as “impact strength” or simply as “impact”, describes the resistance of a material to fracture by a mechanical impact, and is expressed in terms of the amount of energy absorbed by the material before complete fracture. Impact resistance can be measured using, for example, the ASTM D256-06 standard Izod impact testing (also known as “Izod notched impact”, or as “Izod impact”), and/or as described hereinunder, and is expressed as J/m.

As used herein, HDT refers to a temperature at which the respective formulation or combination of formulations deforms under a predetermined load at some certain temperature. Suitable test procedures for determining the HDT of a formulation or combination of formulations are the ASTM D-648 series, particularly the ASTM D-648-06 and ASTM D-648-07 methods. In various exemplary embodiments of the invention the core and shell of the structure differ in their HDT as measured by the ASTM D-648-06 method as well as their HDT as measured by the ASTM D-648-07 method. In some embodiments of the present invention the core and shell of the structure differ in their HDT as measured by any method of the ASTM D-648 series. In the majority of the examples herein, HDT at a pressure of 0.45 MPa was used.

Herein, “Tg” of a material refers to glass transition temperature defined as the location of the local maximum of the E″ curve, where E″ is the loss modulus of the material as a function of the temperature.

Broadly speaking, as the temperature is raised within a range of temperatures containing the Tg temperature, the state of a material, particularly a polymeric material, gradually changes from a glassy state into a rubbery state.

Herein, “Tg range” is a temperature range at which the E″ value is at least half its value (e.g., can be up to its value) at the Tg temperature as defined above.

Without wishing to be bound to any particular theory, it is assumed that the state of a polymeric material gradually changes from the glassy state into the rubbery within the Tg range as defined above. The lowest temperature of the Tg range is referred to herein as Tg (low) and the highest temperature of the Tg range is referred to herein as Tg (high).

Herein throughout, whenever a curable material is defined by a property of a hardened material obtained therefrom, it is to be understood that this property is for a hardened material obtained from this curable material per se.

By “Tensile strength” it is meant the maximum stress that a material can withstand while being stretched or pulled before breaking. Tensile strength may be determined, for example, according to ASTM D-638-03.

By “Tensile modulus” it is meant the stiffness of a material, defined as the relationship between stress (force per unit area) and strain (proportional deformation) in a material in the linear elasticity regime of a uniaxial deformation. Tensile modulus may be determined, for example, according to ASTM D-638-04.

By “flexural strength” or “flexural stress” it is meant the stress in a material just before it yields in a flexure test. Flexural strength may be determined, for example, according to ASTM D-790-03, unless otherwise indicated.

By “flexural modulus” or “flexural Y modulus” it is meant the ratio of stress to strain in flexural deformation, which is determined from the slope of a stress-strain curve produced by a flexural test such as the ASTM D790. Flexural modulus may be determined, for example, according to ASTM D-790-04, unless otherwise indicated.

According to some embodiments, flexural strength and flexural modulus are determined in accordance to ISO 20795-1 (8.5).

Herein throughout, unless otherwise indicated, viscosity values are provided for a viscosity of a material or a formulation when measured at 25° C. on a Brookfield's viscometer. Measured values are provided in centipoise units, which correspond to mPa/second units.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Design

The present inventors have sought for curable formulations which are usable for additive manufacturing, particularly, 3D inkjet printing, of dental prostheses, including denture artificial teeth, denture base, and, preferably, a monolithic structure that combines denture base and teeth. The present inventors have focused on 3D inkjet printing using a system as described in FIG. 1D, which employs a LED radiation source as a curing energy source and can perform a PolyJet printing process. The PolyJet printing process enables combining different formulations of different colors and/or different mechanical properties, and is particularly useful for manufacturing a full-colored monolithic structure that combines denture base and teeth, and for manufacturing any denture part in a personalized manner.

The present inventors have further sought for such curable formulations which, apart from meeting the 3D-printing process requirements (e.g., exhibit desired viscosity, surface tension, printability, reactivity), meet regulatory dentistry requirements, and particularly, would exhibit biocompatible and mechanical properties as set forth in ISO 20795-1 Dentistry, ISO 10477 Dentistry; and ISO 10993-1 (Biological evaluation of medical devices (for mucosal membrane contact with long term exposure)).

During laborious studies, the present inventors have designed and successfully prepared and practiced modeling and support material formulations that meet the 3D-printing process requirements, the regulatory requirements and the desired color control. All the materials selected for these studies, including curable materials, photoinitiators, dispersants, inhibitors, and coloring agents, were selected as being biocompatible, in accordance with their toxicity profile, also upon post-curing and photobleaching.

The present inventors have recognized that for achieving desirable mechanical properties that meet the ISO 20795-1 and ISO 10477 standards, methacrylate materials of relatively high molecular weight materials, reinforcing materials (fillers), ethoxylated curable materials and multi-functional materials, along with mono-functional curable materials should be used; and have further studied the effect of different materials, for example, dispersants for each coloring agent, inhibitors, and the preparation parameters in order to select materials that would provide desired stability and meet the ISO 10993-1 requirements.

As described in further detail hereinunder, the present inventors have identified formulations that meet all of the above requirements and are usable in the successful manufacturing of full-colored monolithic denture structures.

### Example 2

### Exemplary Modeling Formulations

Table 1 below presents exemplary curable materials and other components that were considered to be included in the modeling material formulations for being used in medical formulations, or for having relatively high NOEAL (no-observed-adverse-effect-level) values.

TABLE 1

| | |
|---|---|
| Component A | Multi-functional (e.g., di-functional) urethane |
| | (meth)acrylate featuring high Tg |
| | Multi-functional (e.g., di-functional) aliphatic |
| | urethane (meth)acrylate featuring Tg higher |
| | than 100° C. |
| Component B | Multi-functional (e.g., di-functional) |
| | (meth)acrylate having high Tg |
| Component B1 | Multi-functional (e.g., di-functional) alicyclic |
| | (meth)acrylate featuring Tg higher than 100° C. |
| Component B2 | Multi-functional (e.g., di-functional) aromatic |
| | (meth)acrylate featuring Tg higher than 200° C. |
| Component C | Filler particles |
| | Silica particles, optionally functionalized by |
| | (meth)acrylic groups |
| Component D | Multi-functional (e.g., di-functional) |
| | ethoxylated (meth)acrylate |
| Component D1 | Multi-functional (e.g., di-functional) |
| | ethoxylated aromatic (meth)acrylate featuring |
| | less than 10 ethoxylated groups and/or Tg that |
| | ranges from 50 to 150° C. |
| Component D2 | Multi-functional (e.g., di-functional) |
| | ethoxylated aromatic (meth)acrylate featuring at |
| | least 10 ethoxylated groups |
| Component E | Mono-functional (meth)acrylate |
| Component E1 | Mono-functional methacrylate |
| Component E2 | Mono-functional acrylate, optionally alicyclic |
| | acrylate |
| Component F | Multi-functional (e.g., tri-functional) |
| | (meth)acrylate |
| Component F1 | Multi-functional (e.g., tri-functional) cyclic |
| | (meth)acrylate (cyanurate) |
| Component F2 | Multi-functional (e.g., tri-functional) aliphatic |
| | (meth)acrylate |
| Component G | Multi-functional (e.g., di-functional) urethane |
| | (meth)acrylate featuring low Tg |
| | Oligomeric multi-functional (e.g., di-functional) |
| | aliphatic urethane (meth)acrylate, having |
| | average MW higher than 1,000 grams/mol and |
| | Tg lower than 100° C. |
| Component H | Dispersant |
| | Dispersant, featuring curable groups (e.g., |
| | oligomeric multi-functional (e.g., di-functional) |
| | aliphatic silicon (meth)acrylate |
| Component I | Inhibitor |
| | FDA-approved inhibitor (e.g., phenol-type) |
| Component J | Photoinitiator |
| Component P | Pigment |
| Component Dp | Pigment's dispersant |

Using various combinations of these components, at varying concentrations (presented in % by weight of the total weight of the formulation), the formulations presented in Table 2 were prepared (e.g., by mixing all the materials at a temperature of up to 50° C.).

TABLE 2

| | I | II | III | IV | V | VI | VII | VIII |
|---|---|---|---|---|---|---|---|---|
| A | 15-25 | 15-25 | 15-25 | 15-25 | 15-25 | 15-25 | X | 15-25 |
| B1 + C | 15-25 | 20-30 | 30-40 | 15-25 | 15-25 | 15-25 | X | X |
| 50:50 | | | | | | | | |
| Mixture | | | | | | | | |
| B1 | X | X | X | X | X | X | 20-30 | 25-35 |
| B2 | X | X | X | X | X | X | 20-30 | X |
| D1 | 5-15 | 5-15 | X | 5-15 | 15-25 | 5-15 | X | X |
| D2 | X | X | X | X | X | X | 5-15 | X |
| E1 | 10-20 | 10-20 | 10-20 | 15-25 | 10-20 | 15-25 | 15-25 | 15-25 |
| E2 | 10-20 | 10-20 | 10-20 | 10-20 | 5-10 | 10-20 | 10-20 | 10-20 |
| F1 | 5-10 | 5-10 | X | X | X | X | X | X |
| F2 | X | X | X | 5-15 | X | X | X | X |
| G | 5-10 | 1-10 | 5-15 | 5-10 | 5-10 | 5-10 | X | X |
| H | 0.1-1 | 0.1-1 | 0.1-1 | 0.1-1 | 0.1-1 | 0.1-1 | 0.1-1 | 0.1-1 |
| I | √ | √ | √ | √ | √ | √ | √ | √ |
| J | 1-5 | 1-5 | 1-5 | 1-5 | 1-5 | 1-5 | 1-5 | 1-5 |
| P | | 1-5 | | | | | | |
| Dp | | 0.1-1 | | | | | | |

Formulation I is a clear, colorless, optionally transparent, formulation, which contains no pigments. Such a formulation is usable as a base for CMY (cyan magenta and yellow) formulations as described herein.

Formulation II is a white formulation, and the pigment P is a white paste containing 40% of a white pigment in a mixture of curable (meth)acrylate materials.

In addition to the formulations presented in Table 2, the following formulations were casted:

Formulation IX, also referred to herein as WC formulation, containing a mixture of 80% by weight Formulation II and a total of 20% by weight of Formulation I, or of equal weights of (5% wt. each) of Formula I, Formulation X, Formulation XI and Formulation XII.

Formulation X-a cyan formulation, containing a cyan pigment and a respective pigment dispersant as described herein.

Formulation XI-a magenta formulation, containing a magenta pigment and a respective pigment dispersant as described herein.

Formulation XII-a yellow formulation, containing a yellow pigment and a respective pigment dispersant as described herein.

Formulations X, XI and XII have a composition as Formulation I, to which 0.1-0.5% by weight of a paste containing curable materials and 0.05-0.5% by weight of the selected pigment, and a pigment dispersant, is added.

The cyan, magenta and yellow pigments (component P) in all formulations are preferably nanosize-grinded pigments (and not dyes), having an average particle size lower than 1 micrometer, and are selected amongst pigments that exhibit good thermal stability, and are usable in food products and/or medical devices.

The pigment dispersant (component Dp) is preferably a surface active agent approved for food contact printing ink, and is a high molecular weight block copolymer based on a polyurethane chemistry, and which preferably features functional groups that have high affinity to the respective pigment. The pigment dispersant Dp can be the same or different for each pigment.

Table 3 below presents the process parameters of each of the formulations presented in Table 2.

Tables 4A-B below present the mechanical and physical properties required by the ISO 20795-1 standard and measured in accordance therewith, and the respective properties of each of the formulations presented in Table 2.

Table 5 below presents the mechanical and physical properties required by the ISO 10477 standard and measured in accordance therewith, and the respective properties of each of the formulations presented in Table 2.

Table 6 below presents the biocompatibility properties required by the ISO 10993-1 standard and measured in accordance therewith, and the respective properties of each of the formulations presented in Table 2.

TABLE 3

| | | Surface | | |
|---|---|---|---|---|
| | Viscosity | tension | | Stability |
| | (cP @70) | (dyn/cm) | Jettability | (3 weeks @65) |
| Goal | 10-25 | 25-35 | pass | pass |
| I | 19-23 | 28-32 | pass | pass |
| II | 19-23 | 28-32 | pass | pass |
| III | 19-23 | ND | failed | failed |
| IV | failed | ND | failed | failed |
| V | 19-23 | ND | failed | failed |
| VI | 19-23 | ND | pass | pass |
| VII | 19-23 | ND | pass | pass |
| VIII | 19-23 | ND | pass | pas |
| IX | 19-23 | 28-32 | pass | pass |

TABLE 4A

| | I | II | III | IV | V | VI | VII | VIII | Goal |
|---|---|---|---|---|---|---|---|---|---|
| Ult. Flexural | 91.3 ± 0.6 | 88.6 ± 2 | 75.8 ± 0.4 | 2367 ± 138 | N/A | 69 ± 0.9 | 75.4 ± 3 | 66.3 ± 12.5 | >65 |
| Strength | | | | | | | | | |
| (MPa) | | | | | | | | | |
| Flexural | 2728 ± 33 | 2856 ± 48 | 2121 ± 58 | 80.6 ± 9 | N/A | 1920 ± 96.6 | 2021 ± 70 | 1866 ± 157 | >2000 |
| modulus | | | | | | | | | |
| (Mpa) | | | | | | | | | |
| Water sorption | 32 | 28.8 | | | | | | | <32 |
| (μg/mm³) | | | | | | | | | |
| Water | 1.12 | 1.6 | | | | | | | <1.6 |
| solubility | | | | | | | | | |
| (μg/mm³) | | | | | | | | | |
| Surface | Pass | Pass | | | | | | | Pass |
| characteristics | | | | | | | | | |
| Color | Pass | Pass | Pass | Too | Too | Pass | Too | Pass | Pass |
| | | | | dark | dark | | dark | | |
| Translucency | Pass | Pass | | | | | | | Pass |
| Freedom from | Pass | Pass | | | | | | | Pass |
| porosity | | | | | | | | | |
| Shade | Pass | Pass | | | | | | | Pass |
| consistency | | | | | | | | | |

TABLE 4B

| | IX | X | XI | XII | Goal |
|---|---|---|---|---|---|
| Ult. Flexural | 86.4 ± 1 | 82 ± 1 | 76 ± 1.6 | 76 ± 2.2 | >65 |
| Strength (MPa) | | | | | |
| Flexural | 2726 ± 101 | | | | >2000 |
| modulus (MPa) | | | | | |
| Water | 27 | | | | <32 |
| sorption | | | | | |
| (μg/mm³) | | | | | |
| Water | −1.4 | | | | <1.6 |
| solubility | | | | | |
| (μg/mm³) | | | | | |
| Surface | Pass | | | | Pass |
| characteristics | | | | | |
| Color | Pass | | | | Pass |
| Translucency | Pass | | | | Pass |
| Freedom from | Pass | | | | Pass |
| porosity | | | | | |
| Shade | Pass | | | | Pass |
| consistency | | | | | |

The following can be deduced from the data presented in Tables 3, 4A and 4B:
When a mixture of components B1 and C is used in an amount higher than 25% wt., or, when a silica filler is used in an amount higher than 15% wt., as in Formula III, process parameters are not met (see, Table 3);
When component D1 is used in an amount higher than 15% wt., as in Formulation V, process parameters and color are adversely affected;
In the absence of component C and/or component A, as in Formulations VII and VIII, the mechanical properties required by ISO 20795-1 are not met. Adding component B1 improves mechanical strength but results in darkening of the color, as in Formulation VII;
Using component F2 instead of F1, as in Formulation IV, mechanical properties are improved but process parameters are not met and color is darkened.

Formulations I, II and IX were therefore considered as meeting the process and ISO 20795-1 requirements and were further characterized for meeting the requirements of ISO 10477 and ISO 10993-1, as follows.

TABLE 5

| | I | II | IX | Goal |
|---|---|---|---|---|
| Ult. Flexural | 91.3 ± 0.6 | 88.6 ± 2 | >95 | >50 |
| Strength (MPa) | | | | |
| Water sorption | 32 | 28.8 | 27 | <40 |
| (μg/mm³) | | | | |
| Water solubility | 1.12 | 1.6 | −1.4 | <7.5 |
| (μg/mm³) | | | | |
| Depth of cure | Pass | Pass | Pass | Pass |
| Color stability | Pass | Pass | Pass | Pass |
| Shade consistency | Pass | Pass | Pass | Pass |
| Surface finish | Pass | Pass | Pass | Pass |

As can be seen, all formulations meet the requirements of ISO 10477.

TABLE 6

| | I | II | IX | Goal |
|---|---|---|---|---|
| Cytotoxicity | Pass | Pass | Pass | Pass |
| Sensitization | Pass | Pass | Pass | Pass |
| Irritation | Pass | Pass | Pass | Pass |
| Acute systemic | Pass | Pass | Pass | Pass |
| Genotoxicity | Pass | Pass | Pass | Pass |
| Toxicity/Carcinogenicity | Pass | Pass | Pass | Pass |
| (for EU) | | | | |
| Implantation | Pass | Pass | Pass | Pass |

As can be seen, all formulations meet the requirements of ISO 10993-1.

FIG. 4 is a photograph of an exemplary monolithic structure of a denture base and artificial teeth, prepared by 3D inkjet printing using an AM system for example as depicted in FIG. 1D, and the exemplary white, transparent and colored formulations as described herein. As can be seen, a monolithic structure with accurate control of the colors of each part of the structure was successfully prepared.

### Example 3

### Stability

Additional tests were conducted in order to evaluate the stability of the selected formulations over time.

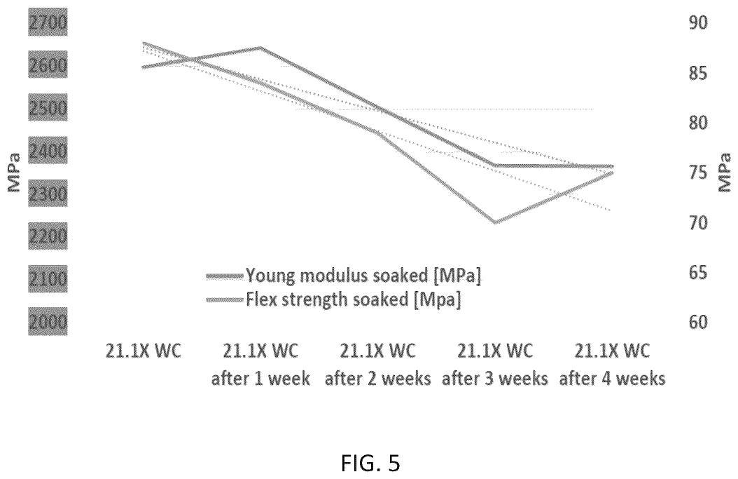

FIG. 5 presents comparative plots showing the change in the mechanical properties of an object prepared using Formulation IX over one month in water at 37° C., in accordance with ISO 20795-1, demonstrating a change of less than 10%, which meets the ISO 20795-1 standard requirements.

Table 7 below presents the mechanical properties of an object prepared using Formulation IX before and after subjecting the object to mercury lamp irradiation for 2 hours, over one month in water at 37° C., in accordance with ISO 20795-1, demonstrating a change of less than 10%, which meets the ISO 20795-1 standard requirements, showing a minor and even nullified change in the mechanical properties, thereby meeting the ISO 20795-1 standard requirements.

TABLE 7

| | Young Modulus [MPa] | Flex Strength [MPa] |
|---|---|---|
| T = 0 | 2597 ± 46 | 88 ± 1.5 |
| T = after 2 hours mercury | 2670 ± 145 | 89 ± 2.3 |
| lamp irradiation | | |

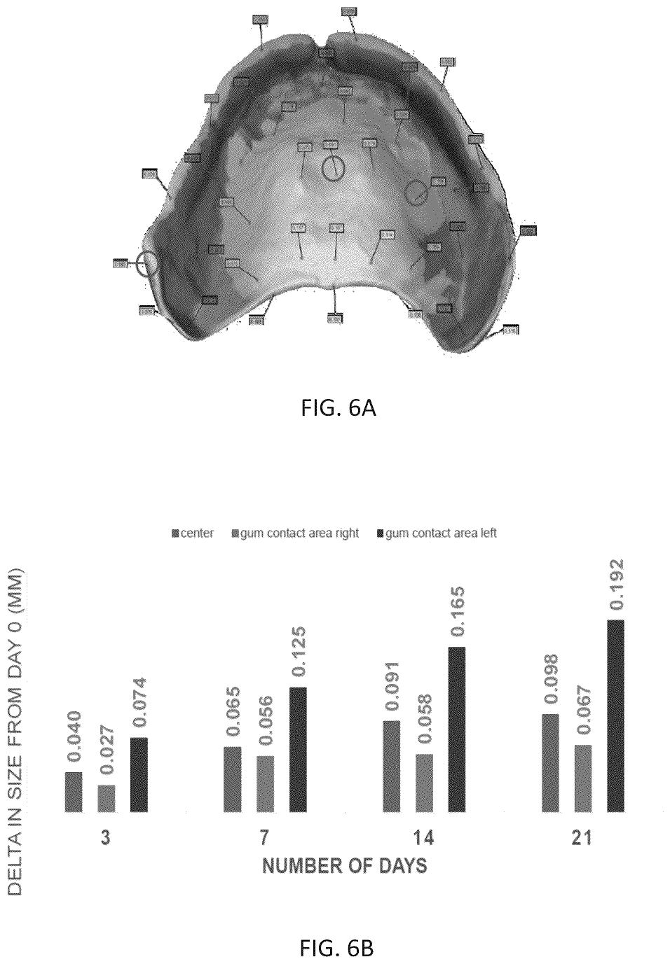

FIGS. 6A-B present dimensional changes of an internal portion (printed in a matte mode as described herein) of an exemplary monolithic denture structure prepared using at least some of Formulations I, II, IX, X, XI and XII upon immersion in water at 37° C. FIG. 6B is a bar graph showing the measured dimensions of exemplary points in the central portion of the denture and in the left and right gum contact areas, as marked by circles in FIG. 6A.

A change of no more than 200 micrometers was observed upon soaking in water for 3 weeks, in accordance with the requirements of ISO 20795-1.

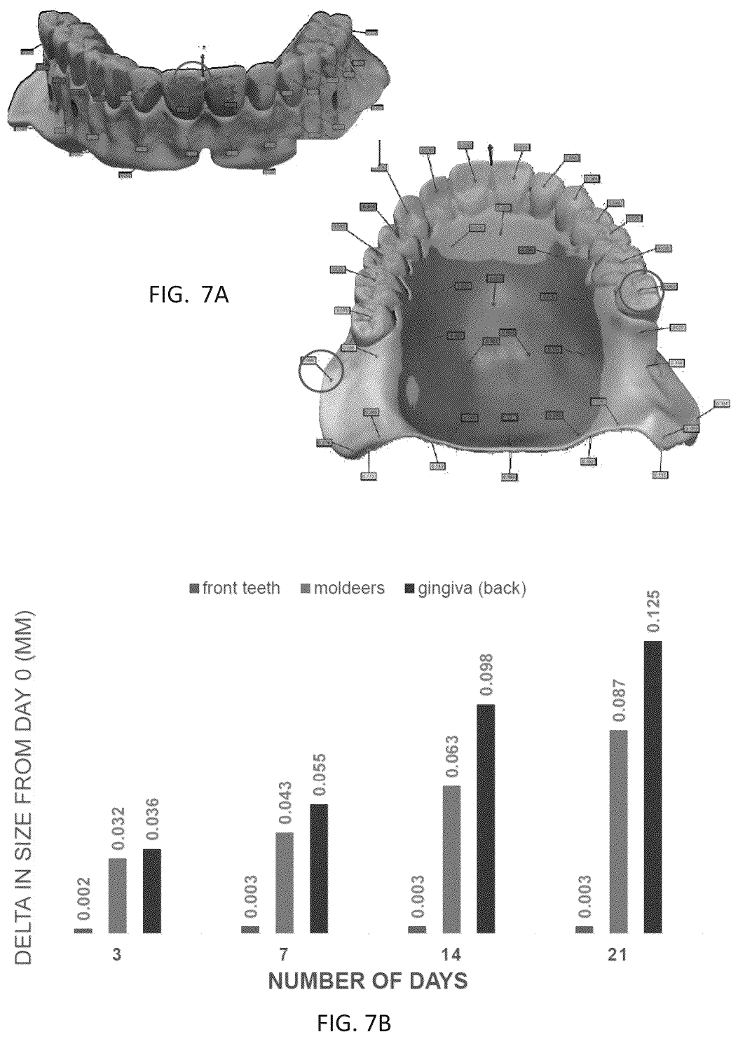

FIGS. 7A-B present dimensional changes of an external portion (printed in a glossy mode) of an exemplary monolithic denture structure prepared using at least some of Formulations I, II, IX, X, XI and XII as described herein, upon immersion in water at 37° C. FIG. 7B is a bar graph showing the measured dimensions of exemplary upper tooth, as marked by a circle in FIG. 7A upper image, and of exemplary points of the molders and gingiva, as marked by circles in FIG. 7A lower image.

A change of no more than 125 micrometers in the outskirts and 3 microns in the teeth was observed upon soaking in water for 3 weeks, in accordance with the requirements of ISO 20795-1.

The stability of the formulations upon storage was tested by measuring a change in the viscosity at 70° C. during 21 days at 65° C. The obtained data is presented in Table 8 below and show no substantial change in the viscosity, indicating the chemical stability of the formulations.

TABLE 8

| | Viscosity (day 0) | Viscosity after 21 days at |
|---|---|---|
| Formulation | [centipoises] | 65° C. [centipoises] |
| I | 20.4 | 20.5 |
| II | 19.4 | 19.5 |
| X | 20 | 20.4 |
| XI | 19.9 | 20.1 |
| Support | 16.3 | 16.3 |

### Example 4

### Support Material Formulation

The present inventors have uncovered that currently available support material formulations do not perform optimally in the context of the denture structures and have designed and successfully practiced accordingly novel formulations that are usable for providing a support material. The support material formulations are usable in combination with any of the modeling material formulations according to the present embodiments. More specifically, the present inventors have identified a need to include in the support material formulation a multi-functional curable material, in additional to hydrophilic mono-functional curable materials commonly used in such formulation.

An exemplary support material formulation according to some embodiments of the present invention comprises:

A hydrophilic mono-functional (meth)acrylate, preferably PEGylated acrylate such as PEA6; 15-25% by weight;

Hydrophilic mono-functional (meth)acrylamide such as HEAA; 10-20% by weight;

Multi-functional (e.g., di-functional) aliphatic/alicyclic (meth)acrylate; 1-5% by weight; and

Non-curable water-soluble or water-immiscible polymeric material as described herein (e.g., a polyol or a mixture of polyols); 40-60% by weight.

The formulation preferably further comprises a photoinitiator, at a concentration of 0.1-1% by weight, and optionally an inhibitor and/or a surfactant.

The stability of an exemplary formulation is shown in Table 8 hereinabove.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A denture structure, comprising:
at least one artificial tooth; and
a base structure supporting said artificial tooth;
wherein said artificial tooth and said base structure are made of materials having different colors and are monolithically connected.

2. The denture structure of claim 1, wherein said tooth is multicolored.

3. The denture structure of claim 1, wherein said base structure is multicolored.

4. The denture structure of claim 1, wherein each of said tooth and said base structure is multicolored.

5. The denture structure of claim 1, wherein said tooth is colored digitally.

6. The denture structure of claim 1, wherein said base structure is colored digitally.

7. The denture structure of claim 1, wherein said tooth is a printed tooth and said base structure is a printed base structure.

8. The denture structure of claim 1, comprising silica.

9. The denture structure of claim 1, wherein mechanical properties of the denture structure are in accordance with the requirements of at least one of: ISO 20795-1 and ISO 10477.

10. The denture structure of claim 1, wherein biocompatibility properties of the denture structure are in accordance with ISO 10993-1.

11. A method of fabricating a denture structure by additive manufacturing, comprising:
dispensing a plurality of building material formulations to form a layer corresponding to cross-sectional shapes of at least one artificial tooth and a base structure; and
hardening said layer to form a monolithic structure having said cross-sectional shapes of said tooth and said base structure, wherein said cross-sectional shapes of said artificial tooth and said base structure have different colors.

12. The method of claim 11, wherein said dispensing comprises dispensing building material formulations of different colors to form said cross-sectional shape of said tooth.

13. The method of claim 11, wherein said dispensing comprises dispensing building material formulations of different colors to form said cross-sectional shape of said base structure.

14. The method of claim 11, wherein said dispensing comprises dispensing building material formulations of different colors to form each of said cross-sectional shapes of said tooth and said base structure.

15. The method of claim 11, wherein said dispensing is executed to form a digital color for said cross-sectional shape of said tooth.

16. The method of claim 11, wherein said dispensing is executed to form a digital color for said cross-sectional shape of said base structure.

17. The method of claim 11, wherein at least one of said building material formulations comprises silica.

18. The method of claim 11, comprising repeating said dispensing and said hardening to form a subsequent layer, wherein a combination of building material formulations dispensed to form said cross-sectional shape of said tooth in said layer is different from a combination of building material formulations dispensed to form said cross-sectional shape of said tooth in said subsequent layer.

19. The method of claim 18, comprising repeating said dispensing and said hardening to form a subsequent layer, wherein a combination of building material formulations dispensed to form said cross-sectional shape of said base structure in said layer is different from a combination of building material formulations dispensed to form said cross-sectional shape of said base structure in said subsequent layer.

20. The method of claim 11, comprising repeating said dispensing and said hardening to form a subsequent layer, wherein a combination of building material formulations dispensed to form said cross-sectional shape of said base structure in said layer is different from a combination of building material formulations dispensed to form said cross-sectional shape of said base structure in said subsequent layer.
